(19) United States
(12) Patent Application Publication
(10) Pub. No.: US 2012/0087864 A1
(43) Pub. Date: Apr. 12, 2012

(54) METHOD FOR DIAGNOSING AND/OR EVALUATING RETINAL DISEASE

(75) Inventors: Yukihiko MASHIMA, Tokyo-to (JP); Ryuji UENO, Montgomery, MD (US)

(73) Assignee: R-TECH UENO, LTD., Tokyo (JP)

(21) Appl. No.: 13/271,620

(22) Filed: Oct. 12, 2011

Related U.S. Application Data
(63) Continuation-in-part of application No. 13/084,927, filed on Apr. 12, 2011.
(60) Provisional application No. 61/408,237, filed on Oct. 29, 2010.
(60) Provisional application No. 61/362,945, filed on Jul. 9, 2010.
(60) Provisional application No. 61/326,811, filed on Apr. 22, 2010.
(60) Provisional application No. 61/323,342, filed on Apr. 12, 2010.
(60) Provisional application No. 61/323,338, filed on Apr. 12, 2010.

(51) Int. Cl. A61K 49/00; G06F 19/00; A61B 3/00; A61B 3/024; A61K 38/06; A61P 27/02

(52) U.S. Cl. 424/9.2; 514/20.8; 351/246; 351/224; 702/19

(57) ABSTRACT

The present application provides a method for diagnosing and/or evaluating the presence or absence, severity or degree of the improvement of a retinal disease in a subject, which comprises determining and/or evaluating circulatory parameters, retinal function, retina morphology and/or visual relating quality of life (QOL).

### CROSS REFERENCE TO RELATED APPLICATIONS

This application is a continuation in part of U.S. application Ser. No. 13/084,927 that claims priorities to U.S. Provisional Patent Application No. 61/323,338 filed Apr. 12, 2010, U.S. Provisional Patent Application No. 61/323,342 filed Apr. 12, 2010, U.S. Provisional Patent Application No. 61/326,811 filed Apr. 22, 2010, U.S. Provisional Patent Application No. 61/362,945 filed Jul. 9, 2010 and U.S. Provisional Patent Application No. 61/408,237 filed Oct. 29, 2010, the contents of each of which are herein incorporated by reference in their entirety.

### BACKGROUND OF THE INVENTION

1. Field of the Invention

The present invention relates to a method for treating retinal diseases using a fatty acid derivative, and the use of an ophthalmic composition comprising the fatty acid derivative. The present invention also relates to a method for improving a visual cell (rod cells and cone cells) function or vision-related quality of life (QOL) of a patient using a fatty acid derivative, and the use of a ophthalmic composition comprising said fatty acid derivative. The present invention further relates to a program and system for evaluating retinal diseases based on retinal sensitivity and vision-related quality of life (QOL).

2. Description of the Related Art

Retina is a membrane-like tissue, which fulfills an important role with respect to a visual function such as reception of light, existing in the innermost layer of eyes. The retina is classified into ten layers, e.g. stratum pigmenti retinae, stratum neuroepitheliale, external limiting membrane, outer granular layer, outer plexiform layer, inner granular layer, inner plexiform layer, ganglion cell layer, nerve fiber layer and internal limiting membrane, formed in this order from the outside. Light irradiated on retina from the outside world transmits the layer of the retina from the internal limiting membrane side and is received by visual cells (rod cells and cone cells) as photoreceptor cells existing in stratum neuroepitheliale. In the visual cells, light is converted into neural signal, and the signal is treated by various nerve cells existing in the retina and information is finally transferred to the cerebral center from ganglion cells existing on a surface (center side of eyeball) of the retina through the optic nerve. The retinal center is the site having a closest relation and seems to be yellowish brown, and is therefore called as the macula area. Furthermore, the central area of the macula lutea is provided with thin retina having a thickness of approximately 0.05 mm and is conically recessed, and is therefore called as conically and is the site with most satisfactory eyesight. Pyramids and rods as light-sensitive receptors of the retina differ in distribution. The pyramids function in the light place and control the light vision, and also a lot of the pyramids exist within a range from the fovea centralis to the macula area and the density decreases when the pyramids apart from the fovea centralis. On the other hand, a lot of rod cells exist around the retina so as to surround the macula area, and also function in the dark place and control scotopic vision.

If disorder arises in visual cell (rod cells and cone cells) function by some kinds of factors, a serious influence is exerted on eyesight, visual field and the like. Examples of one factor which causes visual cell functional disorder include central chorioretinopathy, central chorioretinopathy, hypertensive retinopathy, age-related macular degeneration, arteriosclerotic retinopathy, renal retinopathy, retinopathy diabetic, retinal artery occlusion, retinal vein occlusion, retinal detachment, macular edema, retinitis pigmentosa, prematurity retinopathy, anemic retinopathy, leukemic retinopathy, retinal/choroidal disorders due to external injury, optic neuritis, papilloretinitis, papillitis, neuroretinitis, arachnitis, myelitis, optic nerve atrophy (including diseases associated with optic nerve atrophy, such as Leber's hereditary optic neuropathy (including Lever's disease), optic ischaemic neuropathy, idiopathic optic neuritis, glaucomatous optic neuropathy, optic nerve trauma and others), ocular neovascularization such as choroidal neovascularization and retinal neovascularization, or other retinal diseases such as eyeground diseases. For example, retinitis pigmentosa is a hereditary disease in one of 4,000 to 8,000 persons develops the disease, and also sporadic cases are often found. Histologically, it is a disease based on disorder of a visual cells function in which disorder starts from rods and reaches pyramids. The disease is an intractable disease which starts from night blindness as clinical symptoms and decreases retinal sensitivity to cause visual field constriction and reduced vision, leading to loss of eyesight. Therefore, it is possible to judge that an improvement in the visual cell (rod cells and cone cells) function per se has been recognized if retinal sensitivity (particularly retinal sensitivity of the macula area) of the patient with retinitis pigmentosa is improved.

Fatty acid derivatives are members of class of organic carboxylic acids, which are contained in tissues or organs of human and other mammals, and exhibit a wide range of physiological activities. Some fatty acid derivatives found in nature have, as a general structural property thereof, a prostanoic acid skeleton as shown in the formula (A):

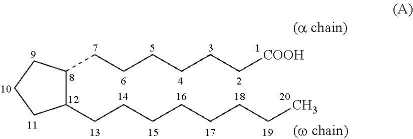

On the other hand, some synthetic Prostaglandin (PG) analogues have modified skeletons. The primary PGs are classified into PGAs, PGBs, PGCs, PGDs, PGEs, PGFs, PGGs, PGHs, PGIs and PGJs on the basis of the structural property of the five membered ring moiety, and further classified into the following three types by the number and position of the unsaturated bond in the carbon chain moiety.

Type 1 (subscript 1): 13,14-unsaturated-15-OH
Type 2 (subscript 2): 5,6- and 13,14-diunsaturated-15-OH
Type 3 (subscript 3): 5,6-, 13,14-, and 17,18-triunsaturated-15-OH.

Further, PGFs are classified on the basis of the configuration of the hydroxy group at the 9-position into a type (wherein the hydroxy group is of the α-configuration) and β type (wherein the hydroxy group is of the β-configuration).

Prostones, having an oxo group at position 15 of prostanoic acid skeleton (15-keto type) and having a single bond between positions 13 and 14 and an oxo group at position 15 (13,14-dihydro-15-keto type)), have been known as substances naturally produced by enzymatic actions during metabolism of the primary PGs and have some therapeutic effect. Prostones have been disclosed in U.S. Pat. Nos. 5,073,569, 5,534,547, 5,225,439, 5,166,174, 5,428,062 5,380,709 5,886,034 6,265,440, 5,106,869, 5,221,763, 5,591,887, 5,770,759 and 5,739,161, the contents of these references are herein incorporated by reference.

Some fatty acid derivatives have been known as drugs used in the ophthalmic field, for example, for lowering intraocular pressure or treating glaucoma. For example, (+)-Isopropyl(Z)-7-[(1R,2R,3R,5S)-3,5-dihydroxy-2-[(3R)-3-hydroxy-5-phenylpentyl]cyclopentyl]-5-heptenoate (general name: latanoprost), Isopropyl (5Z)-7-((1R,2R,3R,5S)-3,5-dihydroxy-2-{(1E,3R)-3-hydroxy-4-[3-(trifluoromethyl)phenoxy]but-1-enyl]cyclopentyl)hept-5-enoate (general name: travoprost), (5Z)-7-{(1R,2R,3R,5S)-3,5-Dihydroxy-2-[(1E,3S)-3-hydroxy-5-phenylpent-1-en-1-yl]cyclopentyl]-N-ethylhept-5-enamide (general name: bimatoprost) and 1-Methylethyl(5Z)-7-{(1R,2R,3R,5S)-2-[(1E)-3,3-difluoro-4-phenoxy-1-butenyl]-3,5-dihydroxy cyclopentyl}-5-heptenoate (general name: tafluprost) have been marketed as ophthalmic solution for the treatment of glaucoma and/or ocular hypertension under the name of Xalatan®, Travatan®, Lumigan® and Tapros®, respectively.

Further, prostones have also been known to be useful in the ophthalmic field, for example, for lowering intraocular pressure and treating glaucoma (see U.S. Pat. Nos. 5,001,153, 5,151,444, 5,166,178, 5,194,429 and 5,236,907), for treating cataract (see U.S. Pat. Nos. 5,212,324 and 5,686,487), for increasing the choroidal blood flow (see U.S. Pat. No. 5,221,690), for treating optic nerve disorder (see U.S. Pat. No. 5,773,471), the contents of these references are herein incorporated by reference. Ophthalmic solution comprising (+)-isopropyl (Z)-7-[(1R,2R,3R,5S)-3,5-dihydroxy-2-(3-oxodecyl)cyclopentyl]hept-5-enoate (general name: isopropyl unoprostone) has been marketed under the name of Rescula® as a pharmaceutical product for the treatment of glaucoma and ocular hypertension. Also, isopropyl unoprostone is known as a BK channel modulator. (Biochimica et Biophysica Acta 1768 (2007) 1083-1092). Documents cited in this paragraph are herein incorporated by reference.

In Japan, several clinical studies for the treatment of patients with retinal pigmentosa using isopropyl unoprostone have been reported. For example, The 50th Annual Congress of Japan Clinical Ophthalmology, 1996; Arch Ophthalmol. vol. 120, 348-352, 2002; The 60th Annual Congress of Japan Clinical Ophthalmology, 2007; IOVS 2008 49 abstract 2185; IOVS 2009 50 abstract 989. Rescula®, an ophthalmic solution approved for the treatment of glaucoma and ocular hypertension in Japan, contains 0.12 w/v of isopropyl unoprostone, the active ingredient with the indication of “instill one drop in an eye twice daily”. To date, all clinical studies conducted on patients with retinal pigmentosa in Japan were conducted according to the dosage regimen of Rescula® ophthalmic solution approved for the treatment of glaucoma and ocular hypertension, i.e. one drop of the ophthalmic solution comprising 0.12 w/v % of the active ingredient, isopropyl unoprostone was instilled in an eye twice daily. In addition, no clinical study include patients treated with placebo ophthalmic solution and therefore, the actual effectiveness of isopropyl unoprostone has not yet evaluated. Documents cited in this paragraph are herein incorporated by reference.

The most standard treatment procedure of ocular diseases is to instill a drug into the eyes. However, generally, it is considered that a drug hardly migrates to the eyeground tissue such as retina in instillation and, if the drug migrates, it is very hard to maintain the concentration of the drug in the tissue. In order to deliver a drug to a tissue in the fundus of the eye, said drug is tried to administer in the vitreous body or to sub-tenon of the eye (US20060286173A, this document is herein incorporated by reference).

### DISCLOSURE OF THE INVENTION

### Problem to be Solved by the Invention

An object of the present invention is to provide a novel method and use of an ophthalmic composition comprising a fatty acid derivative for the treatment of retinal disease. Another object of the present invention is to provide a novel method and use of an ophthalmic composition comprising a fatty acid derivative for improving visual cell (rod cells and cone cells) function in a patient with a retinal disease or for improving the vision-related quality of life (QOL) of a patient.

### Summary of the Invention

The present invention relates to a novel method for the treatment of a retinal disease with a fatty acid derivative and a novel use of a pharmaceutical composition comprising the fatty acid derivative. In particular, the instant invention relates to the method and use of the ophthalmic composition as recited in the claims.

The present invention also relates to a novel method for diagnosing and/or evaluating the presence or absence, severity or degree of the improvement of a retinal disease in a subject, which comprises detecting and/or evaluating circulatory parameters, retinal function, retina morphology and/or visual relating quality of life (QOL). Further, the present invention relates to a method for treating a retinal disease in which the dosing or treatment protocol is adjusted based on the diagnosed and/or evaluated the presence or absence, severity or degree of the improvement of the retinal disease.

The present invention further provides a program and system that can evaluate retinal diseases based on retinal sensitivity and visual-related quality of life (QOL).

The application provides the followings;

(1) An ophthalmic composition comprising a fatty acid derivative for the treatment of a retinal disease in a patient, characterized in that at least two drops at a time of the composition are instilled to an eye of the patient at least twice a day.
(2) The ophthalmic composition of (1), wherein the fatty acid derivative is isopropyl unoprostone.
(3) The ophthalmic composition of (2), wherein the concentration of isopropyl unoprostone in the composition is 0.15%.
(4) The ophthalmic composition of (1), wherein the retinal disease is central chorioretinopathy, central chorioretinitis, hypertensive retinopathy, aged macular degeneration, arterioslerotic retinopathy, renal retinopathy, diabetic retinopathy, retinal artery occlusion, retinal vein occlusion, detachment of the retina, macular edema, retinitis pigmentosa, retinopathy of prematurity, anemic retinopathy, leukemic retinopathy, chorioretinal disorders caused by trauma, optic neuritis, papilloretinitis, papillitis, arachnitis, myelitis, ocular neovascularization or optic atrophy.
(5) The ophthalmic composition of (4), wherein the retinal disease is retinitis pigmentosa.
(6) A method for treating a retinal disease in a patient in need thereof, which comprises instilling at least two drops at a time of an ophthalmic composition comprising a fatty acid derivative to an eye of the patient at least twice a day.
(7) Use of a fatty acid derivative for the preparation of an ophthalmic composition for the treatment of a retinal disease in a patient, characterized in that at least two drops at a time of the composition are instilled to an eye of the patient at least twice a day.
(8) An ophthalmic composition for improving rod cell function and/or cone cell function, comprising a fatty acid derivative as an active ingredient.
(9) The ophthalmic composition of (8), wherein the rod cell function and/or cone cell function is represented by retinal sensitivity.
(10) The ophthalmic composition of (9), wherein retinal sensitivity is that of the central 2 degrees of an ocular fundus determined with a microperimeter MP-1.
(11) An ophthalmic composition comprising a fatty acid derivative for improving rod cell function and/or cone cell function in a patient, characterized in that at least two drops at a time of the composition are instilled to an eye of the patient at least twice a day.
(12) An ophthalmic composition comprising a fatty acid derivative as an active ingredient for improving visual cell function.
(13) The ophthalmic composition of (12), wherein the visual cell function is represented by retinal sensitivity.
(14) The ophthalmic composition of (13), wherein the retinal sensitivity is determined using the central 10-2 SITA standard programs of a Humphrey Field Analyzer.
(15) The ophthalmic composition comprising a fatty acid derivative for improving visual cell function in a patient, characterized in that at least two drops at a time of the composition are instilled to an eye of the patient at least twice a day.
(16) An ophthalmic composition comprising a fatty acid derivative as an active ingredient for improving the vision-related quality of life (QOL) in a subject.
(17) The ophthalmic composition of (16), wherein the vision-related QOL is evaluated with the 25-Item National Eye Institute Visual Functioning Questionnaire (NEI VFQ-25).
(18) The ophthalmic composition of (17), wherein the vision related QOL is evaluated with the vision-related social function (SF) concerning subclass of NEI VFQ-25.
(19) The ophthalmic composition of (16), wherein the subject is a patient with a retinal disease.
(20) The ophthalmic composition of (19), wherein the retinal disease is retinitis pigmentosa.
(21) An ophthalmic composition comprising a fatty acid derivative for improving vision-related quality of life (QOL) in a subject, characterized in that at least two drops at a time of the composition are instilled to an eye of the patient at least twice a day.
(22) The ophthalmic composition of (14), wherein retinal sensitivity is that of the central 2 degrees of an ocular fundus determined with a Humphrey perimeter.
(23) The ophthalmic composition of (1), wherein the composition comprises a fatty acid derivative as an active ingredient and boric acid and/or its salt is for the treatment of a retinal disease.
(24) The ophthalmic composition of (1), wherein the composition comprises a fatty acid derivative as an active ingredient and edetic acid and/or its salt and is for the treatment of a retinal disease.
(25) The ophthalmic composition of (1), wherein the composition comprises a fatty acid derivative as an active ingredient and polysaccharide, and is for the treatment of a retinal disease.
(26) An ophthalmic composition comprising a compound that improves visual function for the treatment of a retinal disease in a patient, characterized in that at least two drops at a time of the composition are instilled to an eye of the patient at least twice a day.
(27) A dosage unit for topical ocular administration for treating a retinal disease in a human patient comprising an effective amount of isopropyl unoprostone and a pharmaceutically suitable excipient, wherein at least three drops of the dosage unit are administered to an eye of the patient per day.
(28) The dosage unit of (27), wherein isopropyl unoprostone is present at a concentration of at least 0.15 w/v %.
(29) The dosage unit of (27), wherein at least four drops of the dosage unit are administered to an eye of the patient per day.
(30) The dosage unit of (27), wherein at least two drops of the dosage unit are administered to an eye of the patient per one time administration, twice a day.
(31) The dosage unit of (27), wherein the dosage unit comprises substantially no benzalkonium chloride.
(32) The dosage unit of (27), wherein the dosage unit is formulated as a sterile unit dose formulation for single use.
(33) The dosage unit of (27), wherein the retinal disease is retinal pigmentosa.
(34) A dosage unit for topical ocular administration for improving visual cell function in a human patient comprising, an effective amount of isopropyl unoprostone and a pharmaceutically suitable excipient, wherein at least three drops of the dosage unit are administered to an eye of the patient per day.
(35) A dosage unit for topical ocular administration for treating retinal degeneration in a human patient comprising, an effective amount of isopropyl unoprostone and a pharmaceutically suitable excipient, wherein at least approximately 72 microgram of isopropyl unoprostone is administered to an eye of the patient per day.
(36) The dosage unit of (35), wherein the isopropyl unoprostone is administered in an amount of at least approximately 90 microgram to an eye of the patient per day.
(37) The dosage unit of (35), wherein the isopropyl unoprostone is administered in an amount of at least approximately 120 microgram to an eye of the patient per day.
(38) The dosage unit of (35), wherein the isopropyl unoprostone is administered in an amount of at least approximately 180 microgram to an eye of the patient per day.
(39) The dosage unit of (35), wherein the dosage unit comprises substantially no benzalkonium chloride.
(40) The dosage unit of (35), wherein the dosage unit is formulated as a sterile unit dose formulation for single use.
(41) The dosage unit of (35), wherein the retinal disease is retinal pigimentosa.
(42) A dosage unit for topical ocular administration for improving visual cell function in a human patient comprising, an effective amount of isopropyl unoprostone and a pharmaceutically suitable excipient, wherein at least approximately 72 microgram of isopropyl unoprostone is administered to an eye of the patient per day.
(43) An ophthalmic composition for topical ocular administration for treating retinal disease in a human patient, wherein at least three drops of the composition are administered to an eye of the patient per day.
(44) The composition of (43), wherein the composition comprises (i) fatty acid derivative as an active ingredient and (ii) a pharmaceutically suitable excipient.
(45) The composition of (44), wherein the fatty acid derivative is isopropyl unoprostone.
(46) The composition of (45), wherein the isopropyl unoprostone is present at a concentration of at least 0.15 w/v %.
(47) The composition of (43) wherein at least four drops of the composition is administered to the patient per day.
(48) The composition of (43), wherein at least two drops of the composition are administered to an eye of the patient per one time administration, twice a day.
(49) The composition of (43), wherein at least two drops of the composition are administered to an eye of the patient per one time administration with at least a 5 minute interval between the drops, twice a day.
(50) The composition of (44), wherein the composition comprises substantially no benzalkonium chloride.
(51) The composition of (43) wherein the composition is formulated as a sterile unit dose formulation for single use.
(52) The composition of (43), wherein the retinal disease is retinal pigimentosa.
(53) An ophthalmic composition for topical ocular administration for improving visual cell function in a human patient, wherein at least three drops of the composition are administered to an eye of the patient per day.
(54) A method for treating retinal disease in a human patient in need of treatment of retinal disease, said method comprising administering at least three drops of an ophthalmic composition comprising an effective amount of an active ingredient topically to an eye of the patient per day.
(55) The method of (54), wherein the composition comprises (i) fatty acid derivative as an active ingredient and (ii) a pharmaceutically suitable excipient.
(56) The method of (55), wherein the fatty acid derivative is isopropyl unoprostone.
(57) The method of (56), wherein the isopropyl unoprostone is present in the ophthalmic composition at a concentration of at least 0.15 w/v %.
(58) The method of (54), wherein at least four drops of the composition are administered to an eye of the patient per day.
(59) The method of (54), wherein at least two drops of the composition are administered to an eye of the patient per one time administration, twice a day.
(60) The method of (54), wherein at least two drops of the composition are administered to an eye of the patient per one time administration with at least a 5 minute interval between drops, twice a day.
(61) The method of (55), wherein the composition comprises substantially no benzalkonium chloride.
(62) The method of (54), wherein the composition is formulated as a sterile unit dose formulation for single use.
(63) The method of (54), wherein the retinal disease is retinal pigimentosa.
(64) A method for improving visual cell function in a human patient in need of improvement of visual cell function, said method comprises administering at least three drops of an ophthalmic composition comprising an effective amount of an active ingredient topically to an eye of the patient per day.
(65) A method for treating retinal disease in a human patient in need of treatment of retinal disease, said method comprises administering to the patient a dosage unit comprising (i) an effective amount of isopropyl unoprostone and (ii) a pharmaceutically suitable excipient, wherein at least approximately 72 microgram of isopropyl unoprostone is administered topically to an eye of the patient per day.
(66) The method of (65), wherein the isopropyl unoprostone is administered in an amount of at least approximately 90 microgram per day.
(67) The method of (65), wherein the isopropyl unoprostone is administered in an amount of at least approximately 120 microgram per day.
(68) The method of (65), wherein the isopropyl unoprostone is administered in an amount of at least approximately 180 microgram per day.
(69) The method of (65), wherein the dosage unit comprises substantially no benzalkonium chloride.
(70) The method of (65), wherein the dosage unit is formulated as a sterile unit dose formulation for single use.
(71) The method of (65), wherein the retinal disease is retinal pigmentosa.
(72) A method for improving visual cell function in a human patient in need of improvement of visual cell function, said method comprises administering to the patient a dosage unit comprising (i) an effective amount of isopropyl unoprostone and (ii) a pharmaceutically suitable excipient, wherein at least approximately 72 microgram of isopropyl unoprostone is administrated topically to an eye of the patient per day.
(73) A method for providing sustained release of an ophthalmic composition comprising a fatty acid derivative and a pharmaceutically acceptable carrier to the back of a human eye, comprising administering an effective amount of an ophthalmic composition topically to the eye of the human patient in need thereof, wherein said method restores or maintains diurnal ocular autonomic function.
(74) The method of (73), wherein the fatty acid derivative comprises isopropyl unoprostone.
(75) A method for providing sustained release of an active ingredient of an ophthalmic composition comprising a fatty acid derivative and a pharmaceutically acceptable carrier to the back of a human eye without causing corneal damage, comprising administering an effective amount of the ophthalmic composition topically to the eye of the human patient in need thereof, wherein said method restores or maintains diurnal ocular autonomic function.
(76) The method of (75), wherein the fatty acid derivative comprises isopropyl unoprostone.
(77) The dosage unit of (27), wherein the isopropyl unoprostone is present at a concentration of at least approximately 0.18 w/v %.
(78) The composition of (45), wherein the isopropyl unoprostone is present at a concentration of at least approximately 0.18 w/v %.
(79) The method of (56), wherein the isopropyl unoprostone is present at a concentration of at least approximately 0.18 w/v %.
(80) The method of anyone of (74) and (76), wherein the fatty acid derivative comprises isopropyl unoprostone present at a concentration of at least approximately 0.18 w/v %.
(81) An ophthalmic composition for topical ocular administration for treating retinal disease in a human patient, wherein at least two drops of the composition are administered to an eye of the patient per one time.
(82) An ophthalmic composition for topical ocular administration for improving visual cell function in a human patient, wherein at least two drops of the composition are administered to an eye of the patient per one time.
(83) A method for treating retinal disease in a human patient in need of treatment of retinal disease, said method comprises ocular locally administering at least two drops of an ophthalmic composition comprising an effective amount of an active ingredient topically to an eye of the patient per one time.
(84) A method for improving visual cell function in a human patient in need of improvement of visual cell function, said method comprises administering at least two drops of an ophthalmic composition comprising an effective amount of an active ingredient topically to an eye of the patient per one time.
(85) The method of any one of (73) and (75), wherein the excipient comprises substantially no benzalkonium chloride.
(86) The method of any one of (73) and (75), wherein the composition is in the form of an ophthalmic solution.
(87) The method of (86), wherein the ophthalmic solution is administered at least three drops to an eye of the patient per day.
(88) The method of (86), wherein the ophthalmic solution is administered at least four drops to an eye of the patient per day.
(89) The method of (86), wherein the ophthalmic solution is administered at least two drops to an eye of the patient per time, twice a day.
(90) The method of (86), wherein the ophthalmic solution is administered at least two drops per time with at least a five minute interval between drops to an eye of the patient, twice a day.
(91) The method of any one of (73) and (75), wherein the composition is in the form of an ophthalmic ointment.
(92) The method of any one of (73) and (75), wherein the composition is administered by injection.
(93) The method of any one of (73) and (75), wherein the composition is administered by an ophthalmic pump.
(94) The method of any one of (73) and (75), wherein the composition is administered by means of contact lens.
(95) The method of any one of (73) and (75), wherein the method treats at least one of retinal pigmentosa, diabetic retinitis, and diabetic retinopathy.
(96) The method of any one of (73) and (75), wherein the step of locally administering comprises using at least one of a cellulose lens, a micropump, a conjunctival pump, an injector, an implantable device, gel capsule, patch, etc.
(97) The method of any one of (73) and (75), wherein the ophthalmic composition comprises at least one of a high viscosity formulation and a gel.
(98) The method of any one of (73) and (75), wherein the ophthalmic composition comprises at least one of an emulsifier, an adsorption enhancer, and an elasticizer.
(99) The method of any one of (73) and (75), wherein the ophthalmic composition provides the sustained release of isopropyl unoprostone to RPE cells.
(100) Any formulation, use, system or device to administer isopropyl unoprostone as a composition of matter in any manner that delivers an Increased Dose.
(101) Administration to a patient in need of a treatment for a neuro-degenerative ophthalmic disease an Increased Dose of isopropyl unoprostone
(102) Use of an endothelin antagonist with an acceptable therapeutic index demonstrated in a human trial in the treatment of a neuro-degenerative ophthalmic disease.
(103) Use of a Microvascular circulation enhancers with an acceptable therapeutic index demonstrated in a human trial in the treatment of a neuro-degenerative ophthalmic disease.
(104) Use of a BK channel modulater with an acceptable therapeutic index demonstrated in a human trial in the treatment of a neuro-degenerative ophthalmic disease.
(105) A method for diagnosing and evaluating the presence or absence, severity or degree of the improvement of a retinal disease in a subject, which comprises determining retinal sensitivity of the subject by the Humphrey visual field test and diagnosing or evaluating presence or absence, severity or degree of the improvement of a retinal disease based on the determined retinal sensitivity.
(106) The method of (105), wherein the retinal sensitivity is determined by the Humphrey visual field test across the central field of the ophthalmic fundus.
(107) The method of (106), wherein the retinal sensitivity of the central 2 degrees of an ocular fundus is determined.
(108) A method for diagnosing and evaluating the presence or absence, severity or degree of the improvement of a retinal disease in a subject, which comprises determining retinal sensitivity across the central area of an ocular fundus of the subject by MP-1 microperimeter and diagnosing or evaluating the presence or absence, severity or degree of the improvement of a retinal disease based on the determined retinal sensitivity.
(109) The method of (108), wherein the retinal sensitivity of the central 2 degrees of an ocular fundus is determined.
(110) A method for diagnosing and evaluating the presence or absence, severity or degree of the improvement of a retinal disease in a subject, which comprises evaluating vision-related quality of life (QOL) of the subject.
(111) The method of (110), wherein the vision related QOL is evaluated with “The 25-Item National Eye Institute Visual Function Questionnaire (NEI VFQ-25)”.
(112) The method of (111), wherein the vision related QOL is evaluated with the vision-related social function (SF)-concerning subclass of NEI VFQ-25.
(113) The method of (110), wherein the subject is a patient with a retinal disease.
(114) The method of any one of (105)-(113), wherein the retinal disease is retinitis pigmentosa.
(115) A program for use with a computer, comprising:

a program instruction for causing a memory of the computer to store a retinal sensitivity in a central area of an ocular fundus of a subject measured by MP-1 microperimeter and/or Humphrey visual field analyzer as stored measurement information; and

a program instruction for causing an evaluation means of the computer to process the stored measurement information and evaluate presence or absence, severity or degree of improvement of a retinal disease in the subject.

(116) The program of (115), wherein the measurement information comprises the retinal sensitivity of the central 10 degrees of the ocular fundus.
(117) The program of (115), wherein the measurement information comprises the retinal sensitivity of the central 2 degrees of the ocular fundus.
(118) A program for use with a computer, comprising:

a program instruction for causing a memory of the computer to store a visual-relating quality of life (QOL) of a subject as stored evaluation information; and

a program instruction for causing an evaluation means of the computer to process the stored evaluation information and evaluate presence or absence, severity or degree of improvement of a retinal disease in the subject.

(119) The program of (118), wherein the vision-related QOL is evaluated with “The 25-Item National Eye Institute Visual Function Questionnaire (NEI VFQ-25)”.
(120) The program of (118), wherein the vision related QOL is evaluated with the vision-related social function (SF)-concerning subclass of NEI VFQ-25.
(121) The program of (118), wherein the subject is a patient with a retinal disease.
(122) The program of any one of (115)-(121), wherein the retinal disease is retinitis pigmentosa.
(123) A system for evaluating the presence or absence, severity or degree of the improvement of a retinal disease in a subject, comprising:

means for storing retinal sensitivity in the central area of an ocular fundus of the subject measured by MP-1 microperimeter and/or Humphrey visual field analyzer as stored measurement information, and

means for processing the stored measurement information and evaluating the presence or absence, severity or degree of the improvement of a retinal disease in the subject.

(124) The system of (123), wherein the measurement information comprises the retinal sensitivity of the central 10 degrees of the ocular fundus.
(125) The system of (123), wherein the measurement information comprises the retinal sensitivity of the central 2 degrees of the ocular fundus.
(126) A system for evaluating the presence or absence, severity or degree of the improvement of a retinal disease in a subject, comprising:

means for storing visual-relating quality of life (QOL) of the subject as stored evaluation information, and

means for processing the stored evaluation information and evaluating the presence or absence, severity or degree of the improvement of a retinal disease in the subject.

(127) The system of (126), wherein the vision-related QOL is evaluated with “The 25-Item National Eye Institute Visual Function Questionnaire (NEI VFQ-25)”.
(128) The system of (127), wherein the vision related QOL is evaluated with the vision-related social function (SF)-concerning subclass of NEI VFQ-25.
(129) The system of (126), wherein the subject is a patient with a retinal disease.
(130) The system of any one of (123)-(129), wherein the retinal disease is retinitis pigmentosa.
(131) A pharmaceutical composition comprising a fatty acid derivative for treating a retinal disease in a patient, which is administered to the patient so that the plasma concentration of the free carboxylic acid metabolite of the fatty acid derivative is 1 ng/ml or more.
(132) The pharmaceutical composition of (131), wherein the fatty acid derivative is isopropyl unoprostone.
(133) A method for treating a retinal disease in a patient, which comprising administering a pharmaceutical composition comprising a fatty acid derivative to the patient so that the plasma concentration of the free carboxylic acid metabolite of the fatty acid derivative is 1 ng/ml or more.
(134) Use of a fatty acid derivative for the preparation of a pharmaceutical composition for the treatment of a retinal disease in a patient, characterized in that the composition is administered to the patient so that the plasma concentration of the free carboxylic acid metabolite of the fatty acid derivative is 1 ng/ml or more.
(135) A pharmaceutical composition comprising a fatty acid derivative for improving visual cell function in a patient, which is administered to the patient so that the plasma concentration of the free carboxylic acid metabolite of the fatty acid derivative is 1 ng/ml or more.
(136) A method for detecting or measuring ocular blood flow in a subject, which comprises the steps of detecting or measuring the temperature of central area of the eyes through Humphrey perimeter or MP-1 microperimeter in the subject.
(137) The method of (136), the central area of the eyes through Humphrey perimeter or MP-1 microperimeter is central 2 degrees.
(138) The method of (136), the central area of the eyes through Humphrey perimeter or MP-1 microperimeter is at least one point of central 4 points.
(139) The method of (136), the ocular blood flow is ocular fundus blood flow.
(140) The method of (139), the ocular fundus blood flow is retinal blood flow or choroidal blood flow.
(141) A method for evaluating the effectiveness of a test compound for causing a thermodynamic change in central area of the eyes through Humphrey visual field analyzer or MP-1 microperimeter in a subject, which comprises:
(i) detecting or measuring a first temperature of central area of eyes of a subject through Humphrey visual field analyzer or MP-1 microperimeter using infrared thermography,
(ii) administering to the subject a composition comprising the test compound,
(iii) detecting or measuring a second temperature of the central area of the eyes of the subject through Humphrey visual field analyzer or MP-1 microperimeter using infrared thermography,
(iv) comparing the first and the second temperatures, and
(v) evaluating the test compound is effective for treating retinal degeneration, when the second temperature is higher than the first temperature
   wherein the first temperature can be taken before and/or after steps (ii) and (iii).
   (142) The method of (141), wherein the infrared thermography is infrared imaging thermography.
   (143) The method of (141), the central area of the eyes through Humphrey perimeter or MP-1 microperimeter is central 2 degrees.
   (144) The method of (141), the central area of the eyes through Humphrey perimeter or MP-1 microperimeter is at least one point of central 4 points.
   (145) A Method for evaluating the effectiveness of a test compound for treating retinal disease, which comprises:
(i) detecting or measuring a first temperature of central area of eyes of a subject through Humphrey visual field analyzer or MP-1 microperimeter using infrared thermography,
(ii) administering to the subject a composition comprising the test compound,
(iii) detecting or measuring a second temperature of the central area of the eyes of the subject through Humphrey visual field analyzer or MP-1 microperimeter using infrared thermography,
(iv) comparing the first and the second temperatures, and
(v) evaluating the test compound is effective for treating retinal degeneration, when the second temperature is higher than the first temperature
   wherein the first temperature can be taken before and/or after steps (ii) and (iii).
   (146) A method for improving visual cell function, comprising: locally administering an effective amount of an ophthalmic composition comprising a fatty acid derivative and a pharmaceutically suitable excipient to a human patient in need thereof, the effective amount of the ophthalmic composition providing a enhanced penetration or sustained release of the fatty acid derivative to a back of an eye, the sustained release being characterized by an AUC value in back-of-the-eye greater than 3 ng/g hr.
   (147) The method of (146), wherein the fatty acid derivative comprises isopropyl unoprostone.
   (148) The method of (146), wherein the AUC value is greater than an AUC value from administration of two drops of 0.12 w/v % isopropyl unoprostone BID.
   (149) The method of (146), wherein the AUC value is greater than an AUC value from administration of less than 72 micrograms of isopropyl unoprostone over 24 hours.
   (150) A method for improving visual cell function, comprising: locally administering an effective amount of an ophthalmic composition comprising a fatty acid derivative and a pharmaceutically suitable excipient to a human patient in need thereof, the ophthalmic composition providing a sustained release of the fatty acid derivative to a back of an eye, the sustained release being characterized by a t1/2 value greater than 1 hr.
   (151) The method of (150), wherein the fatty acid derivative comprises isopropyl unoprostone.
   (152) The method of (151), wherein the t1/2 value is greater than a t1/2 value from administration of two drops of 0.12 w/v % isopropyl unoprostone BID.
   (153) The method of (151), wherein the t1/2 value is greater than a t1/2 value from administration of less than 72 micrograms of isopropyl unoprostone over 24 hours.
   (154) A method for improving visual cell function, comprising: locally administering an effective amount of an ophthalmic composition comprising a fatty acid derivative and a pharmaceutically suitable excipient to a human patient in need thereof, the ophthalmic composition providing a sustained release of the fatty acid derivative to a back of an eye, the sustained release being characterized by a Cmax value greater than 2 ng/g.
   (155) The method of (154), wherein the fatty acid derivative comprises isopropyl unoprostone.
   (156) The method of (155), wherein the Cmax value is greater than a Cmax value from administration of two drops of 0.12 w/v % isopropyl unoprostone BID.
   (157) The method of (155), wherein the Cmax value is greater than a Cmax value from administration of less than 72 micrograms of isopropyl unoprostone over 24 hours.
   (158) An ophthalmic composition capable of sustained release, comprising: an effective amount of a fatty acid derivative and a pharmaceutically acceptable excipient, the composition being capable of providing a sustained release of the fatty acid derivative to a back of an eye when locally administered to a patient in need thereof, the sustained release being characterized by an AUC value greater 3 ng/g hr.
   (159) The ophthalmic composition of (158), wherein the fatty acid derivative comprises isopropyl unoprostone.
   (160) The ophthalmic composition of (159), wherein the sustained release is characterized by an AUC value greater than an AUC value from administration of two drops of 0.12 w/v % isopropyl unoprostone BID.
   (161) The ophthalmic composition of (159), wherein the sustained release is characterized by an AUC value greater than an AUC value from administration of less than 72 micrograms of isopropyl unoprostone over 24 hours.
   (162) The ophthalmic composition of (158), wherein the ophthalmic composition is formulated as a high viscosity formulation.
   (163) The ophthalmic composition of (158), wherein the ophthalmic composition further comprises at least one of an emulsifier, an adsorption enhancer, and an elasticizer.
   (164) An ophthalmic composition capable of sustained release, comprising: an effective amount of a fatty acid derivative and a pharmaceutically acceptable excipient, the composition being capable of providing a sustained release of the fatty acid derivative to a back of an eye when locally administered to a patient in need thereof, the sustained release being characterized by a t1/2 value greater than 1 hr.
   (165) The ophthalmic composition of (164), wherein the fatty acid derivative comprises isopropyl unoprostone.
   (166) The ophthalmic composition of (165), wherein the sustained release is characterized by a t1/2 value greater than a t1/2 value from administration of two drops of 0.12 w/v % isopropyl unoprostone BID.
   (167) The ophthalmic composition of (165), wherein the sustained release is characterized by an AUC value greater than a t1/2 value from administration of less than 72 micrograms of isopropyl unoprostone over 24 hours.
   (168) The ophthalmic composition of (164), wherein the ophthalmic composition is formulated as a high viscosity formulation.
   (169) The ophthalmic composition of (164), wherein the ophthalmic composition further comprises at least one of an emulsifier, an adsorption enhancer, and an elasticizer.
   (170) An ophthalmic composition capable of sustained release, comprising: an effective amount of a fatty acid derivative and a pharmaceutically acceptable excipient, the composition being capable of providing a sustained release of the fatty acid derivative to a back of an eye when locally administered to a patient in need thereof, the sustained release being characterized by a Cmax value greater than 2 ng/g.
   (171) The ophthalmic composition of (170), wherein the fatty acid derivative comprises isopropyl unoprostone.
   (172) The ophthalmic composition of (171), wherein the sustained release is characterized by a t1/2 value greater than a Cmax value from administration of two drops of 0.12 w/v % isopropyl unoprostone BID.
   (173) The ophthalmic composition of (171), wherein the sustained release is characterized by an AUC value greater than a Cmax value from administration of less than 72 micrograms of isopropyl unoprostone over 24 hours.
   (174) The ophthalmic composition of (170), wherein the ophthalmic composition is formulated as a high viscosity formulation.
   (175) The ophthalmic composition of (170), wherein the ophthalmic composition further comprises at least one of an emulsifier, an adsorption enhancer, and an elasticizer.
   (176) Use of amounts of isopropyl unoprostone in the choroid, retinal pigmentary epithelium or other tissues suitable for the promotion of neuroprotection in the eye in amounts that exceed the pharmacodynamically active amounts of isopropyl unoprostone delivered or used in the administration of one drop twice a day dosing of the Registered Formulation (“1 drop BID Dosing”) as tested in the Clinical Trial Experiment wherein any aspect of the aforementioned increase may be measured by increase of either one or more of the following factors: Cₘₐₓ, Cₘᵢₙ, T^{1/2}, AUC) where such increased measure or measures may occur during any part of any therapeutic period (as measured by the period of time during the interval between doses that the amount of isopropyl unoprostone exceeds the Cₘᵢₙ necessary to achieve therapeutic effect) achieved by the 1 drop BID Dosing or any therapeutic period of greater duration achieved by administration of greater amounts of isopropyl unoprostone in a single dose or by extending the number of doses or by releasing a dose over a sustained period of administration (such as by sustained infusion, by micro-pulsed infusion, by transcleral inotophoresis, or by constant elusion of isopropyl unoprostone from a trans-scleral or implanted sustained release delivery formulation or device.) (All of the foregoing referred to hereinafter as “Increased Dose”).
   (177) A computer program for use with a computer, comprising:
(i) a program instruction for causing a first memory to store a first temperature of central area of eyes of a subject, the first temperature being detected or measured through Humphrey visual field analyzer or MP-1 microperimeter using infrared thermography;
(ii) a program instruction for causing a second memory to store a second temperature of the central area of the eyes of the subject, the second temperature being determined or measured after an administration of a composition comprising a test compound to the subject, through Humphrey visual field analyzer or MP-1 microperimeter using infrared thermography;
(iii) a program instruction for causing a process means to calculate and store a difference between the first and second temperatures; and
(iv) a program instruction for causing a process means to evaluate an effectiveness of a test compound for causing a thermodynamic change in central area of the eyes based on the difference:
wherein a determination or measurement of the first temperature is taken before and/or after the determination or measurement of the second temperature.
   (178) The program of (177), wherein the test compound is evaluated to be effective for treating retinal degeneration, when the second temperature is higher than the first temperature.
   (179) A system for evaluating the effectiveness a test compound on ocular blood flow, comprising:
(i) means for storing a first temperature of a central area of eyes of a subject, the first temperature being detected or measured through Humphrey visual field analyzer or MP-1 microperimeter using infrared thermography;
(ii) means for storing a second temperature of the central area of the eyes of the subject, the second temperature being determined or measured after an administration of a composition comprising a test compound to the subject, through Humphrey visual field analyzer or MP-1 microperimeter using infrared thermography;
(iii) means for calculating and storing a difference between the first and second temperatures; and
(iv) means for evaluating an effectiveness of the test compound for causing a thermodynamic change in central area of the eyes based on the difference:
   wherein a determination or measurement of the first temperature (i) is taken before and/or after the determination or measurement of the second temperature.
   (180) The system of (179), wherein the test compound is evaluated to be effective for treating retinal degeneration, when the second temperature is higher than the first temperature.
   (181) A program for use with a computer, comprising:

a program instruction for causing a memory of the computer to store a retinal sensitivity in a central area of an ocular fundus of a subject measured by MP-1 microperimeter and/or Humphrey visual field analyzer as stored measurement information; and

a program instruction for causing an evaluation means of the computer to process the stored measurement information and evaluate the ocular blood flow in the subject.

(182) A system for evaluating the ocular blood flow in a subject which comprises:

means for storing retinal sensitivity in the central area of an ocular fundus of the subject measured by MP-1 microperimeter and/or Humphrey visual field analyzer as stored measurement information; and

means for processing the stored evaluation information and evaluating the ocular blood flow in the patient.

Inventors have found when administering more than the 60 μg of isopropyl unoprostone, such as administering two drops twice a day of Rescula® instead of one drop, twice a day (i.e., either 12 μg/ml 35 μl drop or the 15 μg/ml 22 μdrop), a neuroprotective effect is seen. As described herein below in Test Example 1, delivery of an increased dose of isopropyl unoprostone, provides greater retinal sensitivity as well as an increase in the AUC. This increased dose increases the effective pharmacokinetics or pharmaco-dynamics of the previous formulation, including one or more of an improved dosing period; an increase in the AUC; an increase in the volume and/or improved distribution of the isopropyl unoprostone in and around the eye, including the anterior (e.g., the surface and anterior chamber), the medial, and the posterior segment (i.e., the retina choroid); an increase in the Cmax; an increase in the Cmin; and an increase in the concentration and/or improved distribution in the fluids of the eye (e.g., the aqueous humor, blood, the interstitial fluids, the vitreous fluids, and the intracellular fluids).

Especially, the present invention relates to the followings:

(A) A method for diagnosing and/or evaluating the presence or absence, severity or degree of the improvement of a retinal disease in a subject, which comprises determining and/or evaluating circulatory parameters, retinal function, retina morphology, retinal sensitivity and/or visual relating quality of life (QOL).
(B) The method of (A), wherein the determination and/evaluation is performed by using at least one device selected from the group consisting of: Humphrey visual field analyzer (HFA) and Microperimeter-1 (MP-1).
(C) The method of (B), which comprises:

determining retinal sensitivity in the central area of an ocular fundus of the subject by the Humphrey visual field analyzer (HFA) or a Microperimeter (MP-1), and

diagnosing and/or evaluating the presence or absence, severity, or degree of the improvement of the retinal disease based on the determined retinal sensitivity.

(D) The method of (C), wherein retinal sensitivity is determined using one or more of:
central 10 degrees of an ocular fundus determined with micro perimeter MP-1;
central 3 degrees of an ocular fundus determined with micro perimeter MP-1;
central 2 degrees of an ocular fundus determined with micro perimeter MP-1;
central 10 degrees of an ocular fundus determined with Humphrey visual field analyzer;
central 3 degrees of an ocular fundus determined with Humphrey visual field analyzer; and
central 2 degrees of an ocular fundus determined with Humphrey visual field analyzer;
   (E) The method of (A), which comprises:

evaluating visual relating quality of life (QOL) of the subject and

diagnosing and/or evaluating the presence or absence, severity, or degree of the improvement of the retinal disease based on the visual relating quality of life (QOL).

(F) The method of (E), wherein the vision related QOL is evaluated with “The 25-Item National Eye Institute Visual Function Questionnaire (NEI VFQ-25)”.
(G) The method of (E), wherein the visual relating QOL is evaluated with the visual relating social function (SF)-concerning subclass of NEI VFQ-25.
(H) The method of (A), wherein the diagnosis and/or evaluation is conducted by using a computer program for use with a computer, comprising:

a program instruction for causing a memory of the computer to store a information of detected and/or evaluated circulatory parameters, retinal function, retina morphology, retinal sensitivity and/or visual relating quality of life (QOL) of a subject as stored measurement information; and

a program instruction for causing an evaluation means of the computer to process the stored measurement information and evaluate presence or absence, severity or degree of improvement of a retinal disease in the subject.

(I) The method of any one of (B)-(H), which further comprises determining and/or evaluating circulatory parameters, retinal function, retina morphology and/or retinal sensitivity by using at least one device selected from the group consisting of:

### SLO (Scanning Laser Ophthalmoscope),

### Adaptive Optics SLO (AO-SLO),

Adaptive Optics confocal SLO (AO-cSLO),
Fluorescein angiograpy,

### Color Doppler Imaging (CDI),

### Laser Doppler Flowmetry (LDF),

Scanning laser Doppler flowmetry (SLDF),
Scanning laser polarimetry (SLP),
Spectral domain optical coherence tomography (SD-OCT),

### Retina Functional Imager (RFI),

### Tonometry (OBF),

Pulsatile ocular blood flowmetry (POBF),

### Oxymeter,

Visual field analyzer,

### Perimetry

### Heidelberg Edge Perimeter (HEP),

### Electroretinography (ERG),

Multifocal Electroretinography (mfERG) and

### OCT (Optical Coherence Tomography).

(J) A method for evaluating the effectiveness of a test compound for the treatment of retinal disease, which comprises:

(i) detecting and/or evaluating circulatory parameters, retinal function, retina morphology, retinal sensitivity and/or visual relating quality of life (QOL) of the subject to obtain a baseline value,

(ii) administering to the subject a composition comprising a test compound,

(iii) detecting and/or evaluating circulatory parameters, retinal function, retina morphology and/or visual relating quality of life (QOL) of the subject after the administration of the composition comprising a test compound to obtain a test value, and

(iv) comparing the baseline value to the test value, wherein the difference between the baseline value and test value indicates the effectiveness of the test compound.

(K) A method for treating a retinal disease in a subject, comprising:

measuring the central area of an ocular fundus in a subject to determine one or more of circulatory parameters, retinal function, retina morphology and retinal sensitivity,

diagnosing and/or evaluating the presence, severity, or degree of the improvement of the retinal disease based on the determined circulatory parameters, retinal function, retina morphology and/or retinal sensitivity in the subject, and

adjusting the dosing or treatment protocol based on the presence, severity, or degree of the improvement of the retinal disease.

(L) The method of (J), wherein retinal sensitivity in the central area of an ocular fundus of the subject is determined using at least central 2 degrees, and the presence, severity, or degree of the improvement of the retinal disease is diagnosed and/or evaluated based on the determined retinal sensitivity in the subject.
(M) The method of (K), wherein the presence, severity, or degree of the improvement of the retinal disease is diagnosed and/or evaluated based on one or more of the circulatory parameters, retinal function and retina morphology, in addition to the retinal sensitivity in the subject.
(N) The method of (J), which further comprising:

evaluating visual relating quality of life (QOL) of the subject,

wherein the presence, severity, or degree of the improvement of the retinal disease is determined and/or evaluated based on the QOL in addition to one or more of the circulatory parameters, retinal function, retina morphology and retinal sensitivity in the subject.

For the treatment of retinal degeneration and retinal diseases, known indications typically have “U” shaped response curves such that the outcome and effect will improve only up to a point, and after that point, the response and effects decline and will often have a neurotoxic effect. For example, nitric oxide (“NO”) plays a beneficial role in neurotransmission and vasodilatation at low doses, while at higher concentrations, it is implicated in the pathogenesis of stroke, demyelination, and other neurodegenerative diseases. R. N. Saha and K. Pahan, Antioxidants & Redox Signaling, Vol. 8, No. 5 & 6, 929 (2006). Similarly, many NMDA receptor antagonists used for treating ocular disease induce neurotoxicity at higher doses (Wlaz et al., Eur. J. Neurosci. 1994; 6:1710-1719).

Surprisingly, the Inventors have found that, by increasing the effective does of isopropyl unoprostone, a neuroprotective effect is seen that provides preservation of neuronal function. Thus, improvements in one or more of cellular function, cellular neuroprotection, cellular survival, cellular nutrition, cellular oxygen supply, cellular waste excretion (e.g., the retina to the choroid), intra-ocular pressure lowering, aqueous humor outflow facility (so as to lower intra-ocular fluid volume), and blood and aqueous humor vessel flow potential are found. This increased dosage is particularly useful for the treatment of neuro-degenerative ophthalmic diseases such as glaucoma, age-related macular degeneration (AMD), and retinitis pigmentosa.

### BRIEF DESCRIPTION OF DRAWINGS

The present invention will become more fully understood from the detailed description and the accompanying drawings, wherein:

FIG. 1 is a graph showing the change of Humphrey MD value over time observed in Test Example 1. (Transition of change value of Humphrey MD value [dB]) The “change values” shown in the graph represent the changes from the value observed before the treatment. Patients with retinal pigmentosa were received 0.15% isopropyl unoprostone ophthalmic solution or placebo twice daily. ♦ two drops per one time group, ▪ one drop per one time group, and ▴ placebo group;

FIG. 2 is a graph showing the change of VFQ-25 subscale “vision-related social function” after 24 weeks treatment. (Between-group comparison of change value of VFQ-25 subscale “vision-related social function” (after 24 weeks)) The “change values” shown in the graph represent the changes from the value determined before the treatment;

FIG. 3 is a graph showing the change of VFQ-25 total score after 24 weeks treatment. (Between-group comparison of change value of VFQ25 total score (after 24 weeks)) The “change values” shown in the graph represent the changes from the value determined before the treatment;

FIG. 4 is a graph showing the value of MP-1 central 2 degrees retinal sensitivity. (Transition of change value of MP-1 central 2 degrees retinal sensitivity) The “change values” shown in the graph represent the changes from the value determined before the treatment;

FIG. 5 is a graph showing the average of the changes of retinal sensitivity observed by MP-1 in central 2 degrees (4 points). (Average retinal sensitivity by MP-1 in central 2 degree (4 points)) The “change values” shown in the graph represent the changes from the value determined before the treatment;

FIG. 6 is a block diagram of a system for evaluating retinal disease according to the invention; and

FIG. 7 is a program flow for evaluating the retinal disease according to the invention.

### DETAILED DESCRIPTION OF THE INSTANT INVENTION

As used herein, “an acceptable therapeutic index” includes the therapeutic index demonstrated in a human trial.

The term “retinitis pigmentosa” refers to a group of genetic retinal diseases wherein there is damage to the retina. Retinitis pigmentosa is a type of retinal dystrophy where abnormalities of the rods, cones and/or retinal pigment epithelium (RPE, the pigmented layer just outside of the retina and attached to the choroid) lead to progressive vision loss.

The term “age-related macular degeneration (AMD)” as used herein is referred to as macular degeneration in an individual over a particular age, such as the age of about 50. In one specific embodiment, it is associated with Drusen and/or thickening of Bruch's membrane. In a particular embodiment of the invention, dark adaptation is one symptom of AMD. In specific embodiments, other degenerations are included in the scope of the term, such as Sorsby's fundus dystrophy.

Endothelin antagonist (ERAs) are compounds that block endothelin receptors. Endothelian antagonists include selective ETA receptor antagonists which affect endothehelin A receptors (i.e., sitaxentan, ambrisentan, atrasentan, and BQ-135); selective ETB receptor antagonists which affect endothelin B receptors and dual antagonists, which affect both receptors (i.e., bosentan, tezosentan)

BK channel modulators are compounds that modulate the Ca(2+) activated K(+) channel and include endogenous BK channel modulators and structural analogues, naturally-occurring BK channel inhibitors and blockers, synthetic BK channel inhibitors and blockers, naturally-occurring BK channel openers and structural analogues, and synthetic BK channel openers.

In some embodiments, local administration is administration of the PG to a portion of the eye, including but not limited to Bruch's membrane, the sclera, the retina, the retinal pigment epithelium, the choroid, the macula, the vitreous, the anterior/posterior chamber and/or in the subretinal space.

The PG compound may be administered via sustained release. Accordingly, this provides a continuous supply of an effective amount of the PG compound to the eye.

In some embodiments, the PG formulation is preferably a high viscosity formulation. As used herein, “high viscosity formulation” means that the viscosity of the formulation is at least 100 centipoise. More preferably, the viscosity is at least 500 centipoises or at least 1000 centipoise. Some examples of high viscosity formulations include gels and ointments. Components that aid in inducing high viscosity include, but are not limited to thickeners, hyaluronic acids, cross-linked hyaluronic acids, crosslinked polymers containing subunits derived from acrylic acid, polyacrylic acids, celluloses derivatives, polycarbophil, polyvinylpyrrolidone, gelatin, dextrin, polysaccharides, polyacrylamide, polyvinyl alcohol, polyvinyl acetate, and derivatives, mixtures and copolymers thereof.

An increased dose of isopropyl unoprostone is shown to have a neuroprotective effect. Thus, isopropyl unoprostone is effective for treating neuro-degenerative ophthalmic diseases. The term “neuro-degenerative ophthalmic disease” includes, for example, glaucoma (all types), Stargardt's disease, age-related macular degeneration (AMD), including both the wet type and dry type, and retinitis pigmentosa.

Accordingly, some embodiments of the invention are directed to uses of prostaglandin compounds in the choroid, retinal pigmentary epithelium and/or other tissues suitable for the promotion of neuroprotection in the eye. The amount can exceed the pharmacodynamically active amounts of a prostaglandin delivered or used in the administration of one drop twice a day dosing of Rescula®. The amount is sufficient to result in an increase in delivery as characterized by any one of Cₘₐₓ, Cₘᵢₙ, T^{1/2}, AUC, or other measures such as the volume of distribution around the eye, or an increase in concentration in the fluids of the eye (i.e., the aqueous humor, the blood, the interstitial fluids, the vitreous fluid, and the intracellular fluid). The increase in measure or measures can occur during any part of any therapeutic period (e.g., as measured by the period of time during the interval between doses that the amount of prostaglandin exceeds the Cₘᵢₙ necessary to achieve therapeutic effect) achieved by the 1 drop BID Dosing of Rescula®. Additionally, the present invention provides that the therapeutic period can be of greater duration, and can be achieved by administration of greater amounts of the prostaglandin such as isopropyl unoprostone in a single dose or by extending the number of doses or by releasing a dose over a sustained period of administration (e.g., such as by sustained infusion, by micro-pulsed infusion, by transcleral iontophoresis, or by constant elusion of the prostaglandin from a trans-scleral or implanted sustained release delivery formulation or device.)

In some embodiments, the increased dose (e.g., at least 72 μg) of isopropyl unoprostone can be measured by increase in delivery to the back of the eye as characterized by any one of Cₘₐₓ, Cₘᵢₙ, T^{1/2}, AUC. In other embodiments, the increased dose of unoprostone can be measured by increase in delivery to the back of the eye as characterized by any one of Cₘₐₓ, Cₘᵢₙ, T^{1/2}, AUC, volume or distribution of isopropyl unoprostone in and around the eye (e.g., the anterior, including the surface and anterior chamber, the medial, and the posterior segment, including the retina choroid); and concentration and distribution in the fluids of the eye (e.g., the aqueous humor, blood, the interstitial fluids, the vitreous fluids, and the intracellular fluids).

The increase in choroidal blood flow can be measured, for example, as described by Reitsamer et al., (Invest Ophthalmol Vis Sci. 2009; 50:2301-7), herein incorporated by reference in its entirety. Vitreous flow can be measured, for example, by fluorophotometry or differential fluorophotometry and can be estimated from, for example, fluorophore decay. Aqueous flow measurements can be measured, for example, by fluorophotometry

When the fatty acid derivatives used herein has a prostanoic acid skeleton, the nomenclature of said fatty acid derivatives used herein is based on the numbering system of prostanoic acid represented in the above formula (A).

The formula (A) shows a basic skeleton of the C-20 fatty acid derivative, but the present invention is not limited to those having the same number of carbon atoms. In the formula (A), the numbering of the carbon atoms which constitute the basic skeleton of the fatty acid derivatives starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (C-1). Similarly, when the number of carbon atoms is decreased in the ω-chain, the number is deleted in the order starting from position 20; and when the number of carbon atoms is increased in the ω-chain, the carbon atoms at the position 21 or later are named as a substituent at position 20. Stereochemistry of the compounds is the same as that of the above formula (A) unless otherwise specified.

In general, each of PGD, PGE and PGF represents a fatty acid derivative having hydroxy groups at positions 9 and/or 11, but in the present specification they also include those having substituents other than the hydroxy groups at positions 9 and/or 11. Such compounds are referred to as 9-deoxy-9-substituted-fatty acid derivatives or 11-deoxy-11-substituted-fatty acid derivatives. A fatty acid derivative having hydrogen in place of the hydroxy group is simply named as 9- or 11-deoxy-fatty acid derivative.

As stated above, the nomenclature of a fatty acid derivative is based on the prostanoic acid skeleton. In the case the compound has similar partial structure as the primary PG, the abbreviation of “PG” may be used. Thus, a fatty acid derivative whose α-chain is extended by two carbon atoms, that is, having 9 carbon atoms in the α-chain is named as 2-decarboxy-2-(2-carboxyethyl)-PG compound. Similarly, a fatty acid derivative having 11 carbon atoms in the α-chain is named as 2-decarboxy-2-(4-carboxybutyl)-PG compound. Further, a fatty acid derivative whose ω-chain is extended by two carbon atoms, that is, having 10 carbon atoms in the ω-chain is named as 20-ethyl-PG compound. These compounds, however, may also be named according to the IUPAC nomenclatures.

Examples of the analogues including substitution compounds or derivatives of the above described fatty acid derivative include a fatty acid derivative whose carboxy group at the end of the alpha chain is esterified; a fatty acid derivative whose α chain is extended, a physiologically acceptable salt thereof, a fatty acid derivative having a double bond between positions 2 and 3 or a triple bond between positions 5 and 6; a fatty acid derivative having substituent(s) on carbon atom(s) at position(s) 3, 5, 6, 16, 17, 18, 19 and/or 20; and a fatty acid derivative having a lower alkyl or a hydroxy (lower) alkyl group at position 9 and/or 11 in place of the hydroxy group.

According to the present invention, preferred substituents on the carbon atom at position(s) 3, 17, 18 and/or 19 include alkyl having 1-4 carbon atoms, especially methyl and ethyl. Preferred substituents on the carbon atom at position 16 include lower alkyls such as methyl and ethyl, hydroxy, halogen atom such as chlorine and fluorine, and aryloxy such as trifluoromethylphenoxy. Preferred substituents on the carbon atom at position 17 include lower alkyl such as methyl and ethyl, hydroxy, halogen atom such as chlorine and fluorine, and aryloxy such as trifluoromethylphenoxy. Preferred substituents on the carbon atom at position 20 include saturated or unsaturated lower alkyl such as C₁₋₄ alkyl, lower alkoxy such as C₁₋₄ alkoxy, and lower alkoxy alkyl such as C₁₋₄ alkoxy-C₁₋₄ alkyl. Preferred substituents on the carbon atom at position 5 include halogen atoms such as chlorine and fluorine. Preferred substituents on the carbon atom at position 6 include an oxo group forming a carbonyl group. Stereochemistry of PGs having hydroxy, lower alkyl or hydroxy(lower)alkyl substituent on the carbon atom at positions 9 and 11 may be α, β or a mixture thereof.

Further, the above described analogues or derivatives may have a ω chain shorter than that of the primary PGs and a substituent such as alkoxy, cycloalkyl, cycloalkyloxy, phenoxy and phenyl at the end of the truncated w-chain.

The fatty acid derivative used in the instant application is represented by the formula (I):

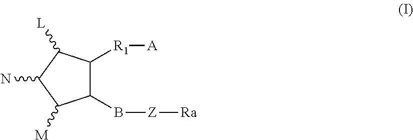

wherein L, M and N are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;

A is —CH₃, —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

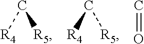

or single bond

wherein, R₄ and R₅ are hydrogen, hydroxy, halogen, lower alkyl, lower alkoxy or hydroxy(lower)alkyl, with the proviso that R₄ and R₅ are not hydroxy and lower alkoxy at the same time,

R₁ is saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is saturated or unsaturated lower or medium bivalent aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; or heterocyclic-oxy group.

A more preferred fatty acid derivative used in the present invention is represented by the formula (II):

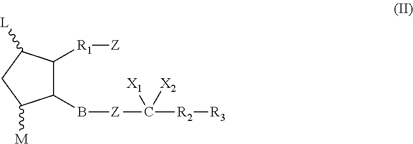

wherein L and M are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;

A is —CH₃, —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

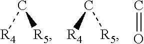

or single bond

wherein, R₄ and R₅ are hydrogen, hydroxy, halogen, lower alkyl, lower alkoxy or hydroxy(lower)alkyl, with the proviso that R₄ and R₅ are not hydroxy and lower alkoxy at the same time;

X₁ and X₂ are hydrogen, lower alkyl, or halogen;

R₁ is saturated or unsaturated bivalent lower or medium aliphatic hydrocarbon residue, which is unsubstituted or substituted with halogen, lower alkyl, hydroxy, oxo, aryl or heterocyclic group, and at least one carbon atom in the aliphatic hydrocarbon is optionally substituted by oxygen, nitrogen or sulfur;

R₂ is single bond or lower alkylene; and

R₃ is lower alkyl, lower alkoxy, lower alkanoyloxy, cyclo(lower)alkyl, cyclo(lower)alkyloxy, aryl, aryloxy, heterocyclic group or heterocyclic-oxy group.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts, preferably pharmaceutically acceptable salts, ethers, esters and amides.

Suitable “pharmaceutically acceptable salts” include salts formed with non-toxic bases conventionally used in pharmaceutical field, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt including such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A means a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo.

Preferred example of A is —COOH, its pharmaceutically acceptable salt, ester or amide thereof.

Preferred example of X₁ and X₂ are hydrogen or halogen, more preferably, both are hydrogen or fluorine atoms at the same time.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur.

Examples of R₁ include, for example, the following groups:

### —CH₂—CH₂—CH₂—CH₂—CH₂—CH₂—,

### —CH₂—CH═CH—CH₂—CH₂—CH₂—,

### —CH₂—CH₂—CH₂—CH₂—CH═CH—,

### —CH₂—C≡C—CH₂—CH₂—CH₂—,

### —CH₂—CH₂—CH₂—CH₂—O—CH₂—,

### —CH₂—CH═CH—CH₂—O—CH₂—,

### —CH₂—C≡C—CH₂—O—CH₂—,

### —CH₂—CH₂—CH₂—CH₂—CH₂—CH₂—CH₂—,

### —CH₂—CH═CH—CH₂—CH₂—CH₂—CH₂—,

### —CH₂—CH₂—CH₂—CH₂—CH₂—CH═CH—,

### —CH₂—C≡C—CH₂—CH₂—CH₂—CH₂—,

### —CH₂—CH₂—CH₂—CH₂—CH₂—CH(CH₃)—CH₂—,

### —CH₂—CH₂—CH₂—CH₂—CH(CH₃)—CH₂—,

### —CH₂—CH₂—CH₂—CH₂—CH₂—CH₂—CH₂—CH₂—,

### —CH₂—CH═CH—CH₂—CH₂—CH₂—CH₂—CH₂—,

### —CH₂—CH₂—CH₂—CH₂—CH₂—CH₂—CH═CH—,

### —CH₂—C≡C—CH₂—CH₂—CH₂—CH₂—CH₂—, and

### —CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains each having one carbon atom.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In this application, a compound wherein the bond between the positions of 13 and 14 is single bond (13,14-dihydro compound) and the 15 position is substituted by keto (═O) may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. The fatty acid derivative of the present invention includes both isomers.

Further, the fatty acid derivatives used in the invention include the bicyclic compound and analogs or derivatives thereof.

The bicyclic compound is represented by the formula (III):

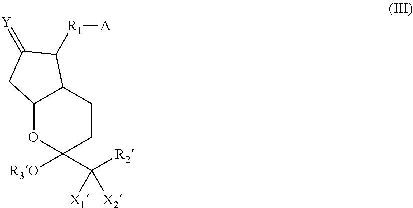

wherein, A is —CH₃, —CH₂OH, —COCH₂OH, —COOH or a functional derivative thereof;

X₁′ and X₂′ are hydrogen, lower alkyl, or halogen;

Y is

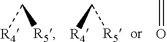

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one carbon atom in the aliphatic hydrocarbon is optionally substituted by oxygen, nitrogen or sulfur;

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group; and

R₃′ is hydrogen, lower alkyl, cyclo(lower)alkyl, aryl or heterocyclic group.

A typical example of fatty acid derivative in this invention is (Z)-7-[(1R,2R,3R,5S)-3,5-dihydroxy-2-(3-oxodecyl)cyclopentyl]hept-5-enoic acid and its derivatives or analogues. The most favorable example fatty acid derivative in this invention is (+)-isopropyl (Z)-7-[(1R,2R,3R,5S)-3,5-dihydroxy-2-(3-oxodecyl)cyclopentyl]hept-5-enoate (hereinafter, isopropyl unoprostone).

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485, the contents of these references are herein incorporated by reference.

The fatty acid derivative described as above is useful for the treatment of retinal diseases. The compound of the present invention is also useful for improving visual cell (rod cell and cone cell) functions or for improving vision-related quality of life (QOL) of a patient.

The term “treatment” or “treating” used herein refers to any means of control of a condition including prevention, cure, relief of the condition, attenuation of the condition and arrest of progression.

Examples of retinal diseases to be treated in the present invention include central chorioretinopathy, central chorioretinopathy, hypertensive retinopathy, age-related macular degeneration, arteriosclerotic retinopathy, renal retinopathy, retinopathy diabetic, retinal artery occlusion, retinal vein occlusion, retinal detachment, macular edema, retinitis pigmentosa, prematurity retinopathy, anemic retinopathy, leukemic retinopathy, retinal/choroidal disorders due to external injury, optic neuritis, papilloretinitis, papillitis, neuroretinitis, arachnitis, myelitis, optic nerve atrophy (including diseases associated with optic nerve atrophy, such as Leber's hereditary optic neuropathy (including Lever's disease), optic ischaemic neuropathy, idiopathic optic neuritis, glaucomatous optic neuropathy, optic nerve trauma and others), neovascularization such as choroidal neovascularization and retinal neovascularization, or other eyeground diseases.

In the present invention, the fatty acid derivative may be formulated into an ophthalmic composition and is administered topically to the eyes of the patient. The ophthalmic composition of the present invention includes any dosage form for topical ocular administration used in the field of ophthalmology, such as an ophthalmic solution, an eye drop and an eye ointment. The ophthalmic composition can be prepared in accordance with conventional means known in the relevant technical field.

The ophthalmic solution or eye drop is prepared by dissolving an active ingredient in a solvent such as an aqueous sterilization solution (for example, brine and buffer solution), or mixing with a powder composition which is dissolved at the time of use. The eye ointment is prepared by mixing an active ingredient with a base.

An “osmotic agent” may added to the ophthalmic composition. The osmotic agent or equivalently an osmoregulating chemical may be any one used usually in the ophthalmology field. Examples of the osmoregulating chemical include, but are not limited to, sodium chloride, potassium chloride, calcium chloride, sodium hydrogen carbonate, sodium carbonate, magnesium sulfate, sodium hydrogen phosphate, sodium dihydrogen phosphate, potassium dihydrogen phosphate, boric acid, borax, sodium hydroxide, hydrochloric acid, mannitol, sorbitol, glucose, glycerin, propylene glycol, polyethylene glycol and the like. The osmoregulating chemical is preferably a sugar alcohol such as mannitol or sorbitol and/or a polyol such as glycerin or propylene glycol.

In the present invention, in order to improve solubility of the fatty acid derivative in the solvent, a solubilizing agent such as a surfactant can be used. The surfactant used in the present invention is not limited as long as it can achieve the object, and a nonionic surfactant is preferred. Examples of the nonionic surfactant include polyoxyethylene sorbitan fatty acid esters such as polyoxyethylene sorbitan monooleate (Polysorbate 80), polyoxyethylene sorbitan monostearate (Polysorbate 60), polyoxyethylene sorbitan monopalmitate (Polysorbate 40), polyoxyethylene sorbitan monolaurate, polyoxyethylene sorbitan trioleate and polyoxyethylene sorbitan tristearate (Polysorbate 65); polyoxyethylene hardened castor oils such as polyoxyethylene hardened castor oil 10, polyoxyethylene hardened castor oil 40, polyoxyethylene hardened castor oil 50 and polyoxyethylene hardened castor oil 60; polyoxyethylene polyoxypropylene glycols such as polyoxyethylene (160) polyoxypropylene (30) glycol [Pluronic F68] and polyoxyethylene (42) polyoxypropylene (67) glycol [Pluronic P123]; polyoxyethylene fatty acid esters such as polyoxyethylene 40 monostearate; and polyoxyethylene alkyl ethers such as polyoxy 10 oleyl ether (Brij 97) and polyoxyl 20 oleyl ether (Brij 98). Preferably, polyoxyethylene sorbitan monooleate (Polysorbate 80), polyoxyethylene hardened castor oil 60, polyoxyethylene 40 monostearate, polyoxyl 10 oleyl ether and the like are exemplified, and these nonionic surfactants may be used alone, or two or more kinds of them may be used in combination.

Furthermore, additive used usually in the field of ophthalmology may be optionally added to the composition of the present invention. Examples of the additive include buffers (for example, boric acid, borax, sodium hydrogen phosphate and sodium dehydrogen phosphate, sodium edetate), preservatives (for example, benzalkonium chloride, benzethonium chloride and chlorobutanol), thickeners (for example, polysaccharides such as sodium hyaluronate, chondroitin sulfate, guar gum, gellan gum, xantan gum and sodium alginate; cellulose polymers such as methyl cellulose, methyl ethyl cellulose and hydroxypropyl methyl cellulose; sodium polyacrylate, a carboxyvinyl polymer and a crosslinked polyacrylic acid.

In the preparation of the eye ointment, the composition may contain, in addition to the above additives, commonly used eye ointment bases. Examples of the eye ointment bases include, but are not limited to, oily bases such as petrolatum, liquid paraffin, polyethylene, Selene 50, Plastibase, macrogol or a combination thereof; emulsion bases containing an oil phase and an aqueous phase emulsified by the surfactant; and water-soluble bases such as hydroxypropyl methyl cellulose, carboxypropyl methyl cellulose and polyethylene glycol.

The term “dosage unit form” and “dosage form” as used herein refer to a single entity for drug administration. In one embodiment, the composition of the present invention may be formulated as a sterile unit dose containing no preservative or substantially free of preservative. The unit dosage form may be administered at one, two, three, four, or more times per day. When ocular local administration is used, one, two, three, four, or more drops may be administered at each time. In one embodiment, the ophthalmic solution is administered at least three drops per day. In another embodiment, the ophthalmic solution is administered at least four drops per day. In another embodiment, the ophthalmic solution is administered at least two drops per time, twice a day. In yet another embodiment, the ophthalmic solution is administered at least two drops per time with at least a five minute interval between drops, twice a day.

In one embodiment, the composition is administered by injection, ophthalmic pump, by means of a contact lens, a cellulose lens, a micropump, a conjunctival pump, an implantable device, a gel capsule, a patch, etc.

The concentration of the fatty acid derivative used in the present invention varies depending on the compounds used, kinds of subjects, age, body weight, symptoms to be treated, desired therapeutic effect, dose, treatment duration and the like, and appropriately proper concentration can be selected.

In the present invention, in the case of usingisopropyl unoprostone, the concentration of the compound is 0.12 w/v % or more, and preferably 0.15 w/v % or more. The upper limit of the concentration is not particularly restrictive and may be set at approximately 10 w/v %.

The method of administrating the ophthalmic composition used in the present invention varies depending on the compounds used, kinds of subject such as animals or humans, age, body weight, symptoms to be treated, desired therapeutic effect, treatment duration and the like. In the case of an ophthalmic solution or eye drop, at least three or more drops may be administered per day. Regarding timing of administration, it is possible to administer with a given interval (for example, every 5 hour) or to administer continuously. In the case of continuously administering, one drop is preferably instilled with at least 5 minute interval after instillation of the previous drop. Preferred dosage regimen includes instillation of at least four or more drops per day. The dosage regimen can be achieved by instilling two or more drops per one administration, twice or more times a day. In this dosage regimen, the second drop is instilled 5 minutes after the instillation of the first drop. In case the number of drops further increases, each drop can also be instilled every 5 minutes. The number of instillations per day is from approximately 2 to 12 times, and the number of drops per one time administration is from two drops to approximately twelve drops.

One drop volume of the ophthalmic composition used in the present invention may be at least approximately 20 μL or more, preferably approximately 30 μL or more, usually approximately from 20 to 50 μL, and preferably approximately from 30 to 40 μL. In the case of using the ophthalmic solution or eye drop of isopropyl unoprostone (0.12 w/v %) in one drop volume of approximately 20 μL, the amount of the compound per one drop required to achieve the object of the present invention is approximately 24 μg or more. It is required to instill approximately 72 μg per day in the case of three drops per day, or approximately 96 μg per day in the case of four drops per day. In the case of using the ophthalmic solution or eye drop of lisopropyl unoprostone (0.15 w/v %) in one drop volume of approximately 20 μL, the amount of the compound per one drop is approximately 30 μg or more. The compound is instilled in the amount of approximately 90 μg per day in the case of three drops per day, and approximately 120 μg per day in the case of four drops per day. In the case of using in one drop volume of approximately 30 μL, the amount of the compound per one drop is approximately 45 μg or more. The compound is instilled in the amount of approximately 135 μg per day in the case of three drops per day, and approximately 180 μg per day in the case of four drops per day.

The term “approximately” used herein can mean plus or minus a range of up to 30%, preferably up to 20%, more preferably up to 10%.

In order to achieve an object of the present invention, the dose of the ophthalmic solution or eye drop per se to be administered per one eye per day also increased as compared with the dose based on the application of the fatty acid derivative typified by isopropyl unoprostone to glaucoma. Therefore, in order to solve the problem of the side effect due to antiseptics such as benzalkonium chloride, an ophthalmic composition substantially free from benzalkonium chloride is preferred in the present invention.

In the present specification, the term “ophthalmic composition substantially free from benzalkonium chloride” and “substantially no benzalkonium chloride” both mean that the composition contains no benzalkonium chloride or the composition contains a given concentration or less of benzalkonium chloride. In the present invention, the concentration of benzalkonium chloride of the ophthalmic composition is less than 0.01 w/v %, preferably 0.005 w/v % or less, and more preferably 0.003 w/v % or less. Also, using a sterile unit dose formulation (for example, one-day disposable or a single dose unit) free from a preservative such as benzalkonium chloride is one of preferred means of the present invention.

Generally, it is considered that a drug hardly migrates to the eyeground tissue such as retina in instillation and, if the drug migrates, it is very hard to maintain the concentration of the drug in the tissue. However, it can be said to be unexpected results that the significant effect is exerted on the treatment of retinal disease even in the topical ocular administration such as instillation by increasing the dose per day of the fatty acid derivative typified by isopropyl unoprostone in the present invention.

Regarding a conventional clinical study on the retinal disease such as retinal pigment degeneration using the fatty acid derivative, any test using a placebo ophthalmic solution as a control is not performed. According to the present invention, definite efficacy of improving a visual cell (rod cells and cone cells) function in the patient with the retinal disease or vision-related quality of life (QOL) of the patient has been recognized for the first time, as the effect of the fatty acid derivative per se typified by isopropyl unoprostone.

In one aspect of the present invention, a method for diagnosing and/or evaluating the presence or absence, severity or degree of the improvement of a retinal disease in a subject, which comprises determining and/or evaluating circulatory parameters, retinal function, retina morphology, retinal sensitivity and/or visual relating quality of life (QOL) is provided. According to the method, the determination and/or evaluation may be conducted by using one or more of the devices as shown below:

### SLO (Scanning Laser Ophthalmoscope),

### Adaptive Optics SLO (AO-SLO),

Adaptive Optics confocal SLO (AO-cSLO),
Fluorescein angiograpy,

### Color Doppler Imaging (CDI),

### Laser Doppler Flowmetry (LDF),

Scanning laser Doppler flowmetry (SLDF),
Scanning laser polarimetry (SLP),
Spectral domain optical coherence tomography (SD-OCT),

### Retina Functional Imager (RFI),

### Tonometry (OBF),

Pulsatile ocular blood flowmetry (POBF),

### Oxymeter,

Visual field analyzer,
Humphrey visual field analyzer (HFA),

### Perimetry,

### Microperimeter-1 (MP-1),

### Heidelberg Edge Perimeter (HEP),

### Electroretinography (ERG),

Multifocal Electroretinography (mfERG) and

### OCT (Optical Coherence Tomography).

In the present invention, it becomes possible to evaluate a visual cell (rod cells and cone cells) function in the patient with the retinal disease in a short period by measuring retinal sensitivity by a micro-visual field test (MP-1) in the central area, for example, central 10 degrees (24 points), particularly a micro-visual field test (MP-1) in central 3 degrees (12 points), preferably a micro-visual field test (MP-1) in central 2 degrees (4 points), and it also becomes possible to diagnose and evaluate the presence or absence of retinal diseases, seriousness and degree of improvement by measuring the retinal sensitivity.

In the present invention, it becomes possible to evaluate a visual cell (rod cells and cone cells) function in a patient with a retinal disease by measuring the retinal sensitivity using a Humphrey visual field test, which has been considered to be insufficient for the evaluation of the visual cell (rod cells and cone cells) function in the patient with the retinal disease and to require evaluation over a long period (of approximately from 3 to 5 years), and also it becomes possible to diagnose and evaluate the presence or absence of retinal diseases, seriousness and degree of improvement by measuring the retinal sensitivity. Particularly, it also becomes possible to evaluate a visual cell (rod cells and cone cells) function in the patient with the retinal disease in a short period (for example, 4 weeks) by using retinal sensitivity (MD value) by a Humphrey visual field test in central 10-2 (central 20 degrees (64 points)).

Furthermore, in the present invention, it becomes apparent that the retinal sensitivity by a micro-visual field test (MP-1) in the central area, for example, central 10 degrees (24 points), particularly central 3 degrees (12 points), preferably central 2 degrees (4 points), correlates with the retinal sensitivity by a Humphrey visual field test in the central area, for example, central 10 degrees (24 points), particularly central 3 degrees (12 points), preferably central 2 degrees (4 points), and it becomes possible to evaluate a visual cell (rod cells and cone cells) function in the patient with the retinal disease in a short period by evaluating the retinal sensitivity by a Humphrey visual field test in the central area, for example, central 10 degrees (24 points), particularly central 3 degrees (12 points), preferably central 2 degrees (4 points), and it also becomes possible to diagnose and evaluate the presence or absence of retinal diseases, seriousness and degree of improvement by measuring the retinal sensitivity.

Retinal sensitivity may be measured at any time after the fatty acid derivative has been administered and treatment has begun. In one embodiment, retinal sensitivity is measured after 4 weeks. In other embodiments, retinal sensitivity is measured after 8 weeks, 12 weeks, or after 24 weeks or more of treatment.

It has been reported that the temperature of the ocular surface correlates with the presence or absence as well as severity of diseases or condition in ocular fundus such as glaucoma (Br. J. Ophthalmol. 2007; 91: 878-881, the contents of this document is herein incorporated by reference). That is, the presence of a retinal disease or the enhancement of severity of a retinal disease in a patient causes decrease of the ocular surface temperature. Accordingly, the present invention also provide a method for detecting or measuring the thermodynamic change of the central area of the eyes by a Humphrey visual field test in the central area, for example, central 10 degrees (24 points), particularly central 3 degrees (12 points), preferably central 2 degrees (4 points), or by a micro-visual field test (MP-1) in the central area, for example, central 10 degrees (24 points), particularly central 3 degrees (12 points), preferably central 2 degrees (4 points). Based on the method, a method of evaluating efficacy of the compound for causing thermodynamic change of the eyes is also provided.

The temperature of the ocular surface can be measured using thermography with an infrared detector. Measurements are taken at the same time every day to avoid bias due to an increase in ocular surface temperature (OST) throughout the day. Preferably, before each examination, room temperature, humidity and air flow are recorded, to make sure to have relatively constant environmental parameters. In one example of this method, the subject is requested to keep the eyes closed for 3-5 s, then to open both eyes wide for each measurement. OST measurements last for 20 s, and the data are registered every second, but only the thermograms taken at the eye opening and at the 20th second after opening (frames 0 and 109, respectively) are evaluated in the statistical analysis. The temperature of five anatomical points across a line running horizontally through the center of the cornea are recorded: the internal canthus (point 1), halfway from the internal canthus and nasal limbus (point 2), the center of the cornea (point 3), halfway from the temporal limbus and external canthus (point 4) and the external canthus (point 5). Student's t test for unpaired data is used to compare the values obtained from the sample population and the background population. In one embodiment, point 3 is preferred as providing the most reliable data.

Also, it is known that the temperature of the ocular surface correlates with the ocular blood flow, namely, the flow rate of ocular blood of the patient with the retinal disease decreases and the temperature of the ocular surface decreases. Therefore, another aspect of the present invention includes a method of detecting or measuring ocular blood flow by detecting or measuring the temperature by a Humphrey visual field test in the central area, for example, central 10 degrees (24 points), particularly central 3 degrees (12 points), preferably central 2 degrees (4 points), or by a micro-visual field test (MP-1) in the central area, for example, central 10 degrees (24 points), particularly central 3 degrees (12 points), preferably central 2 degrees (4 points), and a method of evaluating efficacy of the compound against the retinal disease by the method. In the present invention, the ocular blood flow particularly aims at blood flow of the eyeground as the subject and includes blood flow of the retina and blood flow of the chorioidea.

In the present invention, the method of judging vision-related quality of life (QOL) includes, for example, The 25-Item National Eye Institute Visual Function Questionnaire (NEI VFQ-25), Activities of Daily Vision Scale, vision-specified Sickness Impact Profile (SIP), Medical Outcomes study 12-item short form (SF-12), Medical Outcomes study Short Form 36 item health survey (SF-36), QOL evaluation of retinal pigmentosa and the like. Particularly, The 25-Item National Eye Institute Visual Function Questionnaire (NEI VFQ-25) is preferred, and a subscale constitution suited for evaluation based on NEI VFQ-25 may be appropriately selected and used.

On the other hand, the above results reveal that the same effects as those of the present invention can be expected if it is possible to continuously supply an effective amount of a compound having an action of improving a function of eyeground to the eyeground portion even in the case of topical ocular administration by some means.

Therefore, still another aspect of the present invention is a method of improving the function of eyeground, which is a method for continuously supplying an effective amount of an ophthalmic composition containing a compound having an action of improving a function of eyeground to the eyeground portion by topical ocular administration. Said method restores diurnal ocular autonomic function. An aspect for achieving the object of the present invention is that a conventional dosage regimen of the compound having an action of improving a function of eyeground includes, for example, at least two or more drops per day in the case of instillation of one drop per time once a day, at least three or more drops per day in the case of instillation of one drop per time twice a day, and at least four or more drops per day in the case of instillation of one drop per time three times a day.

Examples of the compound having an action of improving a function of eyeground include, in addition to the fatty acid derivative typified by isopropyl unoprostone, nipradilol and bunazosin hydrochloride each having an ocular blood flow improving action; brimonidine tartrate, ROCK (Rho-kinase) inhibitor (DE-104, K-115, SNJ-1656, etc.), lomerizine hydrochloride, memantine hydrochloride and glutathione each having a neuroprotective function and the like. As long as it is a compound having an action of improving a function of eyeground include, there is no particular limitation.

Examples of means which enables continuous supply of an effective of the compound to the eyeground portion even in the case of topical ocular administration include a gel formulation, lipomas, liposomal, a lipid microemulsion, a microsphere formulation, a nanosphere formulation, an implant formulation and the like by using a thickener. As long as it is means capable of exerting the object of the present invention, it is included in the present invention.

As used herein, “ocular locally administering” includes administration via eye drop, periocular (e.g., subTenon's), subconjunctival, intraocular, subretinal, suprachoroidal and retrobulbar administrations. Ocular local administration may also be administered topically using, for example, an ophthalmic ointment, a gel, a patch, injection, or by means of a contact lens, a cellulose lens, an ophthalmic pump, a micropump, a conjunctival pump, an injector, or an implantable device.

In the clinical test carried out so as to confirm the effects of the present invention, it was recognized that the concentration of a free carboxylic acid of the fatty acid derivative typified by isopropyl unoprostone correlates in plasma with the effect of improving the retinal sensitivity. This means that the treatment of the retinal disease can be effectively performed by administering the fatty acid derivative to the patient so that the concentration of a free carboxylic acid of the fatty acid derivative in plasma becomes a given amount or more.

Therefore, another aspect of the present invention is a method of improving a visual cell function, including administering a fatty acid derivative so that the concentration of a free carboxylic acid of the fatty acid derivative in plasma becomes a given amount or more, and use of a pharmaceutical composition, and is effective for a treatment of the retinal disease.

In the present invention, the concentration of a free carboxylic acid of the fatty acid derivative in plasma is usually 1 ng/mL or more, preferably 2 ng/mL or more, more preferably 2.5 ng/mL or more, and still more preferably 3 ng/mL or more. Blood drawing for the measurement of the concentration of a free carboxylic acid of the fatty acid derivative in plasma may be usually performed within 1 hour after administration of the fatty acid derivative as an active ingredient to the patient, preferably within 30 minutes after administration, and more preferably by around 15 minutes after administration.

According to the present invention, the fatty acid derivative can be systemically or locally applied. Usually, the fatty acid derivative can be administered by topical ocular administration, oral administration, intranasal administration, intraoral administration, administration by inhalation, intravenous injection (including intravenous feeding), subcutaneous injection, endorectal administration, intravaginal administration, percutaneously administration and the like.

The dose can vary depending on the age, body weight, symptoms to be treated, desired therapeutic effect, administration route, treatment duration and the like of the patient. However, in the present invention, the dose may be set so that the concentration of a free carboxylic acid of the fatty acid derivative in plasma becomes a given value (usually 1 ng/mL) or more, and it is also possible to individually set the dose while confirming the concentration in plasma in the patient.

In the present invention, the fatty acid derivative is preferably formulated as a pharmaceutical composition suited for administration by a conventional method. The composition can be those suited for topical ocular administration, oral administration, intranasal administration, intraoral administration, administration by inhalation, injection or perfusion, and external use medicines, suppositories or pessaries.

The pharmaceutical composition of the present invention may further contain physiologically acceptable additives. Examples of the additive include components used together with the compound of the present invention, such as excipients, diluents, extenders, solvents, lubricants, adjuvants, binders, disintegrants, coating agents, encapsulating agents, ointment bases, suppository bases, aerosols, emulsifiers, dispersing agents, suspending agents, thickeners, isotonizing agents, buffers, analgesics, preservatives, antioxidants, taste adjusting agents, aromatics, coloring materials, functional substances (for example, cyclodextrin, biodegradable polymers, etc.), stabilizer and the like. These additives are well known to a person with an ordinary skill in the art, and may be selected from those described in reference books of general pharmaceutics.

The amount of the above-defined fatty acid derivative in the pharmaceutical composition of the present invention may vary depending on the formulation of the composition and can be generally within a range from 0.000001 to 10.0%, more preferably from 0.00001 to 5.0%, and most preferably from 0.0001 to 1%.

Examples of the solid composition for oral administration include tablets, troches, sublingual tablets, capsules, pills, powders, granules and the like. The solid composition may be prepared by mixing one or more active ingredients with at least one inert diluent. The composition may further contain additives other than the inert diluent, for example, lubricants, disintegrants and stabilizers. Tablets and pills may be optionally coated with an enteric-coated or gastric-soluble film. They may be coated with two or more layers. They may be absorbed in a sustained release substance, or microcapsulated. Furthermore, the present composition may be capsulated using an easily decomposable substance such as gelatin. They may be further dissolved in a proper solvent such as fatty acid or a mo-, di- or triglyceride thereof to obtain a soft capsule. In case quick efficacy is required, sublingual tablets may be used.

Examples of the liquid composition for oral administration include emulsions, solutions, suspending agents, syrups, elixirs and the like. The composition may further contain a conventionally used inert diluent, for example, purified water or ethyl alcohol. This composition may contain additives other than the inert diluent, for example, adjuvants such as humectants and suspending agents, sweeteners, flavoring agents, aromatics, preservatives and the like.

The pharmaceutical composition of the present invention may be in the form of a spray composition containing one or more active ingredients, which can be prepared by a known method.

Examples of the intranasal formulation can include aqueous or oily solutions, suspending agents or emulsions each containing one or more active ingredients. In administration by inhalation of active ingredients, the composition of the present invention can be in the form of a suspension, solution or emulsion capable of providing as an aerosol, or in the form of a powder suited for inhalation of a dry powder. The composition for administration by inhalation can further contain propellants which are commonly used.

Examples of the injection composition for parenteral administration of the present invention can include sterilized aqueous or non-aqueous solutions, suspending agents, emulsions and the like. Examples of the diluent for aqueous solutions or suspending agents include distilled water for injection, physiological saline, Ringer's solution and the like.

The non-aqueous diluent for solutions and suspending agents can include, for example, propylene glycol, polyethylene glycol, vegetable oil (olive oil, etc.), alcohol (ethanol, etc.), polysorbate and the like. This composition may further contain additives such as preservatives, humectants, emulsifier and dispersing agents. The composition may be sterilized, for example, by filtering through a bacteria reservation filter, blending a sterilizing agent, or a gas or radioisotope radiation sterilization. The composition for injection can be provided as sterilized powder composition, or can be dissolved in a sterilized solvent for injection before use.

An external use medicine of the present invention includes any external formulation used in the fields of dermatology and otolaryngology, and examples thereof include ointments, creams, lotions, sprays and the like.

Another aspect of the present invention includes suppositories or pessaries, and these can be usually prepared by mixing a commonly used base, for example, cocoa butter which is softened at body temperature, with an active ingredient and a nonionic surfactant having a proper softening temperature suited for an improvement of absorbency may also be used.

According to the present invention, the dose, administration method and dosage form can be set so that the concentration of free carboxylic acid of the fatty acid derivative in plasma in the patient becomes a given value (usually 1 ng/mL) or more, thus making it possible to select treatment strategy suited for the individual patient.

The term “Cmax means Maximum concentration of the drug in the back-of-the-eye tissue (ng/g).

The term “T1/2” means disappearing rate of the drug from the back-of-the eye tissue and Time for 50% reduction of the concentration.

The term “AUC” means Area Under the Curve and Integration of drug concentration by hours.

The present invention will be described in more detail by way of Examples, but the present invention is not limited thereto.

### EXAMPLES

### Formulation Example 1

The respective components were dissolved in purified water so as to adjust to each w/v % shown below, and the solution was aseptically filtered and then filled into a sterilized low density polyethylene container to obtain an ophthalmic solution (one drop volume: approximately 35 μL).

| | |
|---|---|
| 0.15% | isopropyl unoprostone(UF-021) |
| 1.0% | Polyoxyethylene sorbitan monooleate |
| 1.0% | Mannitol |
| 1.9% | Glycerin |
| 0.05% | Sodium edetate |
| 0.003% | Benzalkonium chloride |

### Formulation Example 2

Using the solution prepared by dissolving the respective components in purified water so as to adjust to each w/v % shown below and aseptically filtrating, a sterile unit dose (one-day disposable type) ophthalmic solution was obtained by a Blow Fill Seal system.

| | |
|---|---|
| 0.18% | isopropyl unoprostone |
| 0.70% | Polyoxyethylene sorbitan monooleate |
| 0.30% | Polyoxyl 10 oleyl ether |
| 4.7% | Mannitol |
| 0.01% | Sodium edetate |

### Formulation Example 3

Using the solution prepared by dissolving the respective components in purified water so as to adjust to each w/v % shown below and aseptically filtrating, a sterile unit dose (single unit dose type) ophthalmic solution was obtained by a Blow Fill Seal system.

| | |
|---|---|
| 0.24% | isopropyl unoprostone |
| 0.95% | Polyoxyethylene sorbitan monooleate |
| 0.42% | Polyoxyl 10 oleyl ether |
| 4.7% | Mannitol |
| 0.01% | Sodium edetate |

### Formulation Example 4

Using the solution prepared by dissolving the respective components in purified water so as to adjust to each w/v % shown below and aseptically filtrating, a sterile unit dose (one-day disposable type) ophthalmic solution was obtained by a Blow Fill Seal system.

| | |
|---|---|
| 0.15% | isopropyl unoprostone |
| 1.0% | Polyoxyethylene sorbitan monooleate |
| 1.65% | Boric acid |
| 0.02% | Borax |
| 0.05% | Sodium edetate |

### Formulation Example 5, 6 and 7

Using the solution prepared by dissolving the respective components in purified water so as to adjust to each w/v % shown below and aseptically filtrating, a sterile unit dose (one-day disposable type) ophthalmic solution was obtained by a Blow Fill Seal system.

| | |
|---|---|
| 0.15% | isopropyl unoprostone |
| 1.0% | Polyoxyethylene sorbitan monooleate |
| 1.65% | Boric acid |
| 0.035% | Borax |
| 0.05% | Sodium edetate |
| 0.2, 0.4 or 0.6% | Gellan gum |

### Formulation Example 8

Using the solution prepared by dissolving the respective components in purified water so as to adjust to each w/v % shown below and aseptically filtrating, a sterile unit dose (one-day disposable type) ophthalmic solution was obtained by a Blow Fill Seal system.

| | |
|---|---|
| 0.15% | isopropyl unoprostone |
| 1.0% | Polyoxyethylene sorbitan monooleate |
| 1.65% | Boric acid |
| 0.02% | Borax |
| 0.05% | Sodium edetate |
| 0.6% | Xanthan gum |

### Test Example 1

One drop or two drops of isopropyl unoprostone 0.15 w/v % ophthalmic solution per one time was administered to each eye of patient with retinal pigmentosa daily for 24 weeks, and the following items were examined. 112 patients participated this test.

Change in retinal sensitivity of central 10 degrees (24 points) through an MP-1 microperimeter

Using an combined equipment having a retinal camera and an automatic perimeter, retinal sensitivity of measurement points set preliminarily on the eyeground retina was automatically measured. The feature of MP-1 is that tracking can be automatically performed according to the eye movement, and retinal sensitivity of a local part of the eyeground can be accurately measured by correcting a gap detected during a test. Also, it is possible to serially measure retinal sensitivity at the same site of the eyeground since a test can be performed at the same measurement point of retina as that of the previous test.

Change in retinal sensitivity of central 2 degrees (4 points) through MP-1 microperimeter

Change in retinal sensitivity through Humphrey visual field test (SITA-Standard, 10-2)

Change in retinal sensitivity of central 2 degrees (4 points) through Humphrey visual field test

Change in The 25-Item National Eye Institute Visual Function Questionnaire (NEI VFQ-25) Compo 8:

Change in log MAR eyesight

Change in contrast sensitivity

Findings of optical coherence tomography (OCT) test

Safety evaluation (1. Adverse event, 2. Side effect, 3. Ophthalmologic examination, Vital signs, Clinical examination result)

Concentration of drug in plasma (20 week after the initiation of test drug administration, the concentration of the drug in plasma after 15 minutes had passed since instillation of the second drop). Evaluate items: The concentration of isopropyl unoprostone and its metabolite A (free carboxylic acid of isopropyl unoprostone in plasma was measured.)

The above ophthalmic solution of Formulation Example 1 was used as the test drug. An ophthalmic solution containing the base of Formulation Example 1, which does not contain isopropyl unoprostone, was used as the placebo ophthalmic solution.

Dosage regimen, dose and administration timing in the present test were as follows.

(1) Dosage regimen: Instillation twice* a day, one drop of a first solution was instilled at the time of first instillation and, after 5 minute, one drop of a second solution was instilled into both eyes.

: around 7 o'clock in the morning (6 to 9 o'clock) and around 20 o'clock in the evening (19 to 22 o'clock)

(2) Details of ophthalmic solution

Ophthalmic solution

| Group | First solution | Second solution |
|---|---|---|
| One drop per time group | Placebo | Formulation |
| | | Example 1 |
| Two drops per time group | Formulation | Formulation |
| | Example 1 | Example 1 |
| Placebo group | Placebo | Placebo |

Since subjective symptoms depend on one eye having better visual function than the other, when both eyes satisfy the all selection criteria, the eye having better desimal visual acuity was adopted as the eye for evaluation of efficacy. When both eyes have the same desimal visual acuity, right eye was adopted as the eye for evaluation of efficacy

The results are shown below.

Between-group comparison of the average of Humphrey central 10-2 retina sensitivity (MD value) was made every measurement time point. The results are summarized in Table 1.

TABLE 1
Transition of average of Humphrey central
10-2 retina sensitivity (MD value)

| Measurement | Administered | Number | | Standard |
|---|---|---|---|---|
| time point | group | of cases | Average | deviation |
| 0 week | Placebo group | 33 | −15.026 | 9.859 |
| | One drop per | 38 | −14.116 | 8.766 |
| | time group | | | |
| | Two drops per | 37 | −18.376 | 8.075 |
| | time group | | | |
| After 4 | Placebo group | 32 | −14.803 | 9.352 |
| weeks | One drop per | 37 | −13.952 | 8.323 |
| | time group | | | |
| | Two drops per | 36 | −17.671† | 8.192 |
| | time group | | | |
| After 8 | Placebo group | 31 | −14.999 | 9.513 |
| weeks | One drop per | 38 | −14.074 | 8.251 |
| | time group | | | |
| | Two drops per | 34 | −17.810 | 7.934 |
| | time group | | | |
| After 16 | Placebo group | 33 | −15.138 | 9.395 |
| weeks | One drop per | 36 | −13.768 | 7.572 |
| | time group | | | |
| | Two drops per | 36 | −17.341† | 8.119 |
| | time group | | | |
| After 24 | Placebo group | 31 | −13.871 | 9.013 |
| weeks | One drop per | 38 | −13.699 | 8.126 |
| | time group | | | |
| | Two drops per | 35 | −17.411† | 8.113 |
| | time group | | | |

†t-test, p < 0.05 (versus 0 week)

Transition of the average (MD value) of Humphrey central 10-2 retina sensitivity was compared. As a result, it became apparent that, in the two drops per time group, the average (MD value) of retinal sensitivity increased statistically significantly after 4 weeks, 16 weeks and 24 weeks as compared with the MD value determined at 0 week and, it was concluded that in the two drops per time group, the retinal sensitivity was improved.

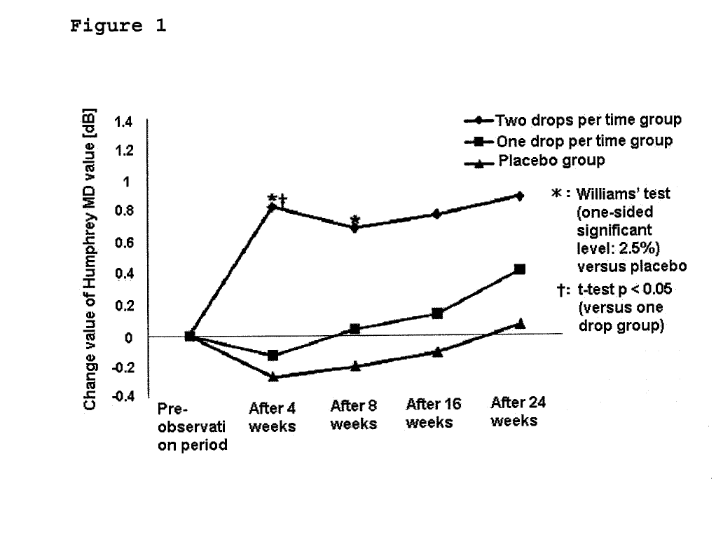

Between-group comparison of the change value of Humphrey central 10-2 retina sensitivity (MD value) was made every measurement time point. The results are summarized in Table 2 and FIG. 1.

TABLE 2
Change value of Humphrey central 10-2
retina sensitivity (MD value)

| Measurement | Administered | Number | | Standard |
|---|---|---|---|---|
| time point | group | of cases | Average | deviation |
| After 4 | Placebo group | 32 | −0.265 | 1.264 |
| weeks | | | | |
| | One drop per | 37 | −0.128 | 1.219 |
| | time group | | | |
| | Two drops per | 36 | 0.831*† | 2.446 |
| | time group | | | |
| After 8 | Placebo group | 31 | −0.202 | 1.123 |
| weeks | One drop per | 38 | 0.042 | 1.225 |
| | time group | | | |
| | Two drops per | 34 | 0.696* | 2.002 |
| | time group | | | |
| After 16 | Placebo group | 33 | −0.112 | 2.153 |
| weeks | One drop per | 36 | 0.136 | 1.6 |
| | time group | | | |
| | Two drops per | 36 | 0.777 | 2.21 |
| | time group | | | |
| After 24 | Placebo group | 31 | 0.066 | 1.036 |
| weeks | One drop per | 38 | 0.417 | 2.133 |
| | time group | | | |
| | Two drops per | 35 | 0.891 | 2.041 |
| | time group | | | |

*Williams' test (one-sided significant level: 2.5%) P < 0.025 (versus placebo group)
†t-test, p < 0.05 (versus one drop per time group)

The value of the change value of Humphrey central 10-2 retina sensitivity (MD value) after 4 weeks was 0.831 in the two drops per time group, −0.218 in the one drop per time group and −0.265 in the placebo group, respectively. As shown in FIG. 1, between-group comparison of the change value of the MD value was made after 4 weeks. As a result, the two drops per time group showed the value which is statistically significantly higher than those of the placebo group and the one drop per time group. The change value of the MD value was compared with that of the placebo group after 8 weeks. As a result, the two drops per time group showed the value which was statistically significantly higher than that of the placebo group. The two drops per time group showed a small variation in the MD value after 4 weeks and, as shown in FIG. 1, the MD value of the one drop per time group and that of the placebo group were far lower than that of the two drops per time group even after 24 weeks. As is apparent from this, a remarkable improvement in retinal sensitivity was observed in a short period of 4 weeks in the two drops per time group, and the effect was maintained for 24 weeks. In contrast, in the one drop per time group, although a tendency of serial improvement was observed as compared with the placebo group, sufficient improvement was not observed even after 24 weeks.

Between-group comparison of the change value of Humphrey central 4 points retinal sensitivity was made every measurement time point. The results are summarized in Table 3.

TABLE 3
Change value of Humphrey central 4 points retinal sensitivity

| Measurement | Administered | Number | | Standard |
|---|---|---|---|---|
| time point | group | of cases | Average | deviation |
| After 4 | Placebo group | 32 | 0.234 | 1.670 |
| weeks | One drop per | 37 | −0.178 | 2.301 |
| | time group | | | |
| | Two drops per | 36 | 0.714 | 3.172 |
| | time group | | | |
| After 8 | Placebo group | 31 | −0.048 | 1.965 |
| weeks | One drop per | 38 | 0.158 | 2.583 |
| | time group | | | |
| | Two drops per | 34 | 0.821 | 3.737 |
| | time group | | | |
| After 16 | Placebo group | 33 | −0.152 | 2.332 |
| weeks | One drop per | 36 | 0.336 | 2.099 |
| | time group | | | |
| | Two drops per | 36 | 1.253*^{†1} | 3.305 |
| | time group | | | |
| After 24 | Placebo group | 31 | 0.539 | 2.366 |
| weeks | One drop per | 38 | 0.334 | 2.877 |
| | time group | | | |
| | Two drops per | 35 | 2.009*^{†1†2} | 2.802 |
| | time group | | | |

*Williams' test (one-sided significant level 2.5% versus placebo group) P < 0.025
^{†1}t-test, p < 0.05 (versus placebo group)
^{†2}t-test, p < 0.05 (versus one drop per time group)

The value of the change value of Humphrey central 4 points retinal sensitivity after 24 weeks was 2.009 in the two drops per time group, 0.334 in the one drop per time group and 0.539 in the placebo group, respectively. As shown in Table 3, between-group comparison of the change value of the retinal sensitivity was made. As a result, the two drops per time group showed the value, which was statistically significantly higher than that of the placebo group, after 16 weeks. Furthermore, the value was statistically significantly higher than that of the placebo group and one drop per time group after 24 weeks.

The retinal sensitivity by a MP-1 central area, particularly central 2 degrees (4 points) correlates with the retinal sensitivity by a Humphrey visual field test in central area, particularly central 2 degrees (4 points). Therefore, it is apparent that the presence or absence of retinal diseases, seriousness, and degree of improvement can be diagnosed and evaluated by evaluating retinal sensitivity by the Humphrey visual field test in central area, particularly central 2 degrees (4 points).

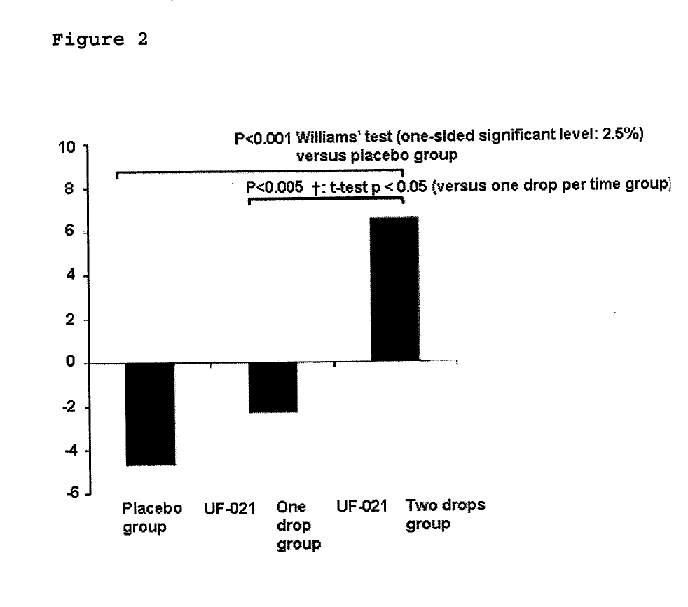

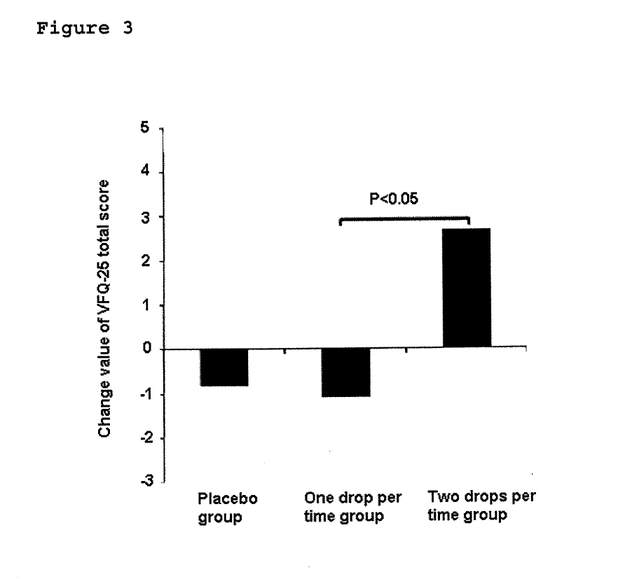

Original NEI VFQ-25 (The 25-item National Eye Institute Visual Function Questionnaire) for evaluation of vision-related QOL of a patient is constituted from visual functions in various living scenes, and 12 subscales (questions of 25 items) for measurement of the degree of restrictions of vision-related physical, mental and social living scenes. In the present test, a questionnaire of Compo 8 constituted from 8 subscales with exception of general health, driving, peripheral vision and ocular pain among the subscales was used. At the time of completion of the pre-observation period (0 week) and completion of the treatment (24 weeks), a change value of score (the value after 24 weeks minus the value at 0 week) was evaluated. Between-group comparison of the change value of VFQ-25 subscale “vision-related social function” is summarized in Table 4 and FIG. 2. Between-group comparison of the change value of the VFQ-25 summary score is summarized in Table 5 and FIG. 3.

TABLE 4
Between-group comparison of change value of VFQ-25 subscale
“vision-related social function” (after 24 weeks)

| Administered | Number | | Standard |
|---|---|---|---|
| group | of cases | Average | deviation |
| Placebo group | 32 | −4.69 | 14.46 |
| One drop per | 38 | −2.3 | 13.89 |
| time group | | | |
| Two drops per | 36 | 6.6*^{†} | 11.76 |
| time group | | | |

*Williams' test (one-sided significant level 2.5% versus placebo group) P < 0.001
^{†}t-test p < 0.005 (versus one drop per time group, versus placebo group)

By between-group comparison of the change value of VFQ-25 subscale “vision-related social function”, in the two drops per time group, significant improvement was recognized as compared with the placebo group and the one drop per time group. This revealed that vision-related QOL of the patient is also improved by an improvement of the retinal sensitivity.

TABLE 5
Between-group comparison of change value
of VFQ-25 summary score (after 24 weeks)

| Administered | Number | | Standard |
|---|---|---|---|
| group | of cases | Average | deviation |
| Placebo group | 32 | −0.83 | 9.56 |
| One drop per | 38 | −1.1 | 8.33 |
| time group | | | |
| Two drops per | 36 | 2.69^{†} | 7.9 |
| time group | | | |

^{†}t-test, p < 0.05 (versus one drop per time group)

Also in a between-group comparison in the change value of VFQ-25 summary score between the case at the time of completion of the pre-observation period (0 week) and the case at the time of completion of the treatment (24 weeks), in the two drops per time group, a significant improvement was recognized as compared with the one drop per time group. Also, as a result of a between-group comparison in VFQ-25 summary score among the placebo group, the one drop per time group and the two drops per time group before the treatment (0 week) and after completion of the treatment (24 weeks), the improvement effect was not recognized in the placebo group and the one drop per time group. However, in the two drops per time group, statistically significant improvement was recognized as compared with the case before treatment (0 week) (p<0.05 (p=0.048 (versus before treatment (0 week) t-test)).

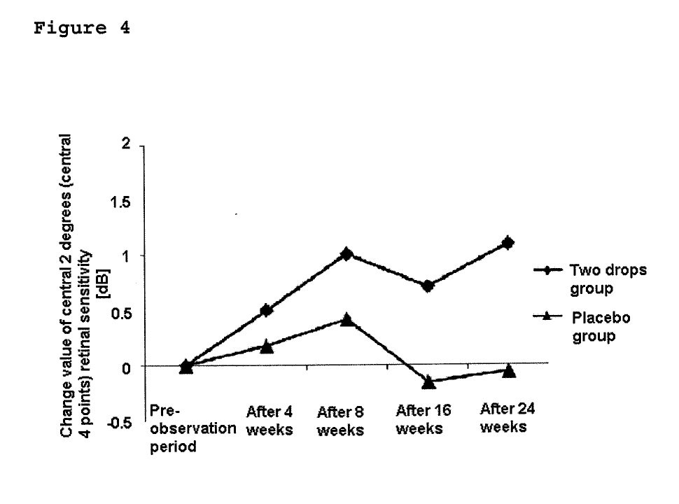

The value of the average retinal sensitivity by MP-1 in central 2 degrees (4 points) changed from the value at the time of completion of the pre-observation period (0 week) at each measurement time point (time of hospital visiting of subject) was calculated in each eye. The results are shown in Table 6 and FIG. 4.

Table 6: Average retinal sensitivity by MP-1 in central 2 degrees (4 points) (transition of change value)

TABLE 6
Average retinal sensitivity by MP-1 in central
2 degrees (4 points) (transition of change value)

| Measurement | Administered | Number | | Standard |
|---|---|---|---|---|
| time point | group | of cases | Average | deviation |
| After 4 | Placebo group | 33 | 0.179 | 2.482 |
| weeks | Two drops per | 38 | 0.5 | 2.23 |
| | time group | | | |
| After 8 | Placebo group | 33 | 0.421 | 3.345 |
| weeks | Two drops per | 38 | 1.013 | 2.434 |
| | time group | | | |
| After 16 | Placebo group | 33 | −0.155 | 3.273 |
| weeks | Two drops per | 38 | 0.713 | 2.838 |
| | time group | | | |
| After 24 | Placebo group | 32 | −0.059 | 3.348 |
| weeks | Two drops per | 35 | 1.1^{†} | 2.786 |
| | time group | | | |

^{†}t-test, p < 0.05 (versus 0 week)

A between-group comparison in average retinal sensitivity by MP-1 in central 2 degrees (4 points) between the placebo group and the two drops per time group before the treatment (0 week) and, after completion of the treatment (24 weeks) was made and, as a result, no improvement effect was recognized in the placebo group. However, in the two drops per time group, statistically significant improvement in retinal sensitivity was recognized as compared with the case before the treatment (0 week) (p<0.05 (p=0.02 corresponding t-test)).

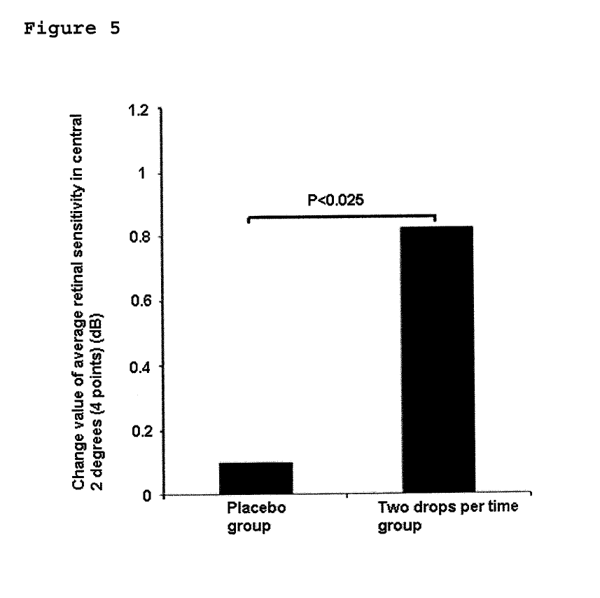

The retinal sensitivity by MP-1 central 2 degrees (4 points) was measured at a pre-observation period, after 4 weeks, after 8 weeks, after 16 weeks and after 24 weeks. It is estimated that variation (error) due to seasonal variation during measuring for 24 weeks may arise. For the purpose of absolutely evaluating the effect of the drug, in order to grasp an overview including negation of both improvement and aggravation, between-group comparison was made by adding up of a difference (change value) with the pre-observation period in four measurements. In the two drops per time group, the change value of retinal sensitivity statistically significantly increased as compared with the placebo group. The results are shown in Table 7 and FIG. 5.

TABLE 7
Average retinal sensitivity by MP-1 in central 2
degrees (4 points) (comparison of change value)

| | Number | | Standard |
|---|---|---|---|
| | of cases | Average | deviation |
| Placebo group | 131 | 0.098 | 3.103 |
| Two drops per | 149 | 0.826* | 2.565 |
| time group | | | |

*Williams' test (one-sided significant level: 2.5% versus placebo group) P < 0.025

As is apparent from the above results, in the two drops per time group, an improvement in retinal sensitivity was significantly recognized as compared with the placebo group.

In average retinal sensitivity by MP-1 in central 2 degrees (4 points), the number of cases of aggravation by 4 dB or more during serial observation for 24 weeks was 7 (21.2%) in the placebo group, 6 (15.8%) in one drop per time group and 1 (2.6%) in the two drops per time group, respectively. In the two drops per time group, the average retinal sensitivity decreases statistically significantly as compared with the placebo group. The results are summarized in Table 8.

TABLE 8
Classification of change value by MP-1 in central 2 degrees
(4 points) average retinal sensitivity (after 24 weeks)

| | | Improvement | Aggravation | |
|---|---|---|---|---|
| | | by 4 dB | by 4 dB | No |
| | Analysis | or more | or more | change |
| | Number of | Number of | Number of | Number of |
| | cases | cases (%) | cases (%) | cases (%) |
| Placebo | 32 | 5 (15.6%) | 7 (21.9%) | 20 (62.5%) |
| group | | | | |
| One drop per | 38 | 3 (7.9%) | 6 (15.8%) | 29 (76.3%) |
| time group | | | | |
| Two drops per | 35 | 6 (17.1%) | 1 (2.9%)* | 28 (80.0%) |
| time group | | | | |

*Likelihood ratio χ2 test, p < 0.05 (versus placebo group)

As is apparent from this, instillation of two drops per time, twice a day significantly suppressed aggravation of the retinal sensitivity by MP-1 in central 2 degrees.

Twenty weeks after the initiation of the test drug administration in Test Example 1, the concentration of the drug in plasma was measured after 15 minutes had passed since instillation of the second drop.

Evaluation item: concentration of metabolite A (free carboxylic acid of isopropyl unoprostone) in plasma
Measuring method: Twenty weeks after initiation of test drug administration, samples were obtained by drawing 4 ml of blood per time after 15 minutes had passed since instillation of the second drop, and then the concentration of the metabolite A in plasma was measured by liquid chromatogram/Tandem mass spectrometer (LC/MS/MS) using a portion of the measuring samples.
Measuring apparatus: liquid chromatograph/Tandem mass spectrometer (LC/MS/MS)
[High-performance liquid chromatography system (SHIMADZU 20A, manufactured by Shimadzu Corporation)
Analysis column: Develosil ODS-UG-3 (2.0 mm I.D.×50 mm, 3 μm, manufactured by Nomura Chemical Co., Ltd.)
Column temperature: 35° C.
Injection amount: 20 μL
Mobile phase A: acetonitrile/10 mmol/L ammonium acetate solution/acetic acid (20:80:0.1, v/v/v)
Mobile phase B: acetonitrile/acetic acid (100:0.1, v/v)
Needle wash: Methanol
Flow rate: 0.25 mL/minute
Mass spectrometry (API 4000, manufactured by Applied Biosystems)]
Ionization method: ESI method (Turbo ion spray, 350° C.)
Internal standard substance: isopropyl unoprostone
Internal standard substance: 17,20-dimethyl PGF_{1α}

In order to evaluate a correlation of the change value (24 weeks) of average retinal sensitivity by MP-1 in central 2 degrees (4 points) with the concentration of metabolite A in plasma of the isopropyl unoprostone instillation group, when the change value (24 weeks) of average retinal sensitivity by MP-1 in central 2 degrees (4 points) was a positive value, it was classified as “improvement”. In contrast, when the change value was zero and a negative value, it was classified as “non-improvement”. Furthermore, each concentration of metabolite A in plasma (1 ng/mL, 2 ng/mL, 2.5 ng/mL and 3 ng/mL) was regarded as a boundary concentration, distribution of the change value (24 weeks) by MP-1 in central 2 degrees (4 points) average retinal sensitivity was confirmed. The results are shown in Table 9.

TABLE 9
Distribution of average retinal sensitivity change
value (24 weeks) by MP-1 in central 2 degrees (4 points)
of concentration of metabolite A in plasma

| | Concentration of compound A in plasma | | | |
|---|---|---|---|---|
| | (ng/mL) | | | |
| Boundary concentration | Less than boundary | | Boundary concentration | |
| 1.0 ng/mL | concentration | | or more | |
| Number of improved | 11 | (50.0%) | 33 | (64.7%) |
| cases (ratio) | | | | |
| Number of non-improved | 11 | (50.0%) | 18 | (35.3%) |
| cases (ratio) | | | | |
| Number of total cases | 22 | (100.0%) | 51 | (100.0%) |
| (ratio) | | | | |
| Boundary concentration | Less than boundary | | Boundary concentration | |
| 2.0 ng/mL | concentration | | or more | |
| Number of improved | 28 | (57.1%) | 16 | (66.7%) |
| cases (ratio) | | | | |
| Number of non-improved | 21 | (42.9%) | 8 | (33.3%) |
| cases (ratio) | | | | |
| Number of total cases | 49 | (100.0%) | 24 | (100.0%) |
| (ratio) | | | | |
| Boundary concentration | Less than boundary | | Boundary concentration | |
| 2.5 ng/mL | concentration | | or more | |
| Number of improved | 32 | (54.2%) | 12 | (85.7%) |
| cases (ratio) | | | | |
| Number of non-improved | 27 | (45.8%) | 2 | (14.3%) |
| cases (ratio) | | | | |
| Number of total cases | 59 | (100.0%) | 14 | (100.0%) |
| (ratio) | | | | |
| Boundary concentration | Less than boundary | | Boundary concentration | |
| 3.0 ng/mL | concentration | | or more | |
| Number of improved | 35 | (55.6%) | 9 | (90.0%) |
| cases (ratio) | | | | |
| Number of non-improved | 28 | (44.4%) | 1 | (10.0%) |
| cases (ratio) | | | | |
| Number of total cases | 63 | (100.0%) | 10 | (100%) |
| (ratio) | | | | |

*Likelihood ratio χ2 test, p < 0.05

As the boundary concentration of the concentration of metabolite A in plasma increased, the degree of the change in average retinal sensitivity by MP-1 in central 2 degrees (4 points) was improved. Particularly, when the boundary concentration of the concentration of metabolite A in plasma was 2.5 ng/mL or more, the change value of the average retinal sensitivity by MP-1 in central 2 degrees (4 points) was significantly improved.

A list of items of the side effect confirmed until completion of medication (24 weeks) in Test Example 1, and the number of appearance cases and the appearance ratio of the side effect of each group are shown in Table 10.

TABLE 10
SIDE EFFECT LIST

| | Placebo group | One drop per time | Two drops per time |
|---|---|---|---|
| | (Versus number of | (Versus number of | (Versus number of |
| | cases: 35 cases) | cases: 39 cases) | cases: 38 cases) |
| | Number of | Number of | Number of |
| Symptoms | appearance cases | appearance cases | appearance cases |
| All side effects* | 12 (34.3) | 28 (71.8) | 21 (55.3) |
| Eye abnormality | 0 (0.0) | 0 (0.0) | 1 (2.6) |
| Dry eye | 1 (2.9) | 1 (2.6) | 0 (0.0) |
| Eye irritation | 0 (0.0) | 9 (23.1) | 4 (10.5) |
| Eye swelling | 0 (0.0) | 1 (2.6) | 0 (0.0) |
| Macular edema | 0 (0.0) | 1 (2.6) | 0 (0.0) |
| Bloodshot eyes | 0 (0.0) | 1 (2.6) | 0 (0.0) |
| Punctate keratitis | 6 (17.1) | 4 (10.3) | 4 (10.5) |
| Macular hole | 1 (2.9) | 0 (0.0) | 0 (0.0) |
| Eye pruritus | 0 (0.0) | 1 (2.6) | 1 (2.6) |
| Bloodshot | 0 (0.0) | 0 (0.0) | 1 (2.6) |
| Hypertrichosis | 0 (0.0) | 0 (0.0) | 1 (2.6) |
| Irritation of applied site*^{†} | 4 (11.4) | 13 (33.3) | 13 (34.2) |
| Foreign body sensation | 0 (0.0) | 1 (2.6) | 0 (0.0) |

*Dose reactivity (Conchan-Armitage test), p < 0.05
^{†}3 group comparison (Fisher's direct probability calculation method), p < 0.05

In any group, no serious side effect arised, and the number of appearance cases of all side effects was 12 (34.3%) in the placebo group, 28 (71.8%) in the one drop per time group, and 21 (55.3%) in the two drops per time group, respectively. The drug administration group showed significantly higher side effect as compared with the placebo group, but most of side effects were mild. In details of the side effect, the one drop per time group and the two drops per time group caused significantly higher irritation at the time of instillation as compared with the placebo group, but the difference between the one drop per time group and the two drops per time group was not recognized. As is apparent from this, a dose-dependent correlation between an improvement in retinal sensitivity and the appearance of the side effect due to frequent instillation was not recognized.

A decrease of intraocular pressure (IOP) was seen in the placebo group, the one drop per time group, and the two drop per time group over the 24-week measurement period. However, the one drop and two drop groups saw a greater decrease in IOP over this time period as compared to the placebo.

The presence or absence of retinal diseases, seriousness, or degree of improvement described above can be evaluated by using a retinal disease evaluation system including a computer. In this case, the retinal disease evaluation system preferably includes a memory or storage means for storing retinal sensitivity of the central area, measured through a microperimeter (MP-1) and/or a Humphrey perimeter, as measurement information; evaluation unit or evaluation means for processing the measurement information stored in the above storage means, and evaluating the presence or absence of retinal diseases, seriousness, or degree of improvement; and display or output means for outputting the evaluation results of the above evaluation means. The evaluation means processes the measurement information according to evaluation items (the presence or absence of retinal diseases, seriousness, degree of improvement) using a program stored in the computer. Also, the above measurement information preferably includes retinal sensitivity in central 10 degrees (24 points), and particularly preferably retinal sensitivity in central 2 degrees (4 points).

The retinal disease evaluation system of another aspect preferably includes storage means for storing vision-related quality of life (QOL) as evaluation information; evaluation means for processing the evaluation information stored in the above storage means, and evaluating the presence or absence of retinal diseases, seriousness, or degree of improvement; and output means for outputting the evaluation results of the above evaluation means. The above vision-related quality of life is preferably measured using “The 25-Item National Eye Institute Visual Function Questionnaire (NEI VFQ-25)” as a measure. Alternatively, the above vision-related quality of life may be measured using “The 25-Item National Eye Institute Visual Function Questionnaire (NEI VFQ-25)” subscale “vision-related social function (Social function: SF)” as a measure. Alternatively, the above vision-related quality of life may be vision-related quality of life of the patient with retinal diseases.

The retinal disease evaluation system preferably includes a retinal diseases evaluation program which enables a computer to function as storage means for storing retinal sensitivity of the central area measured through a microperimeter (MP-1) as measurement information, and evaluation means for processing the measurement information stored in the above storage means, and evaluating the presence or absence of retinal diseases, seriousness, or degree of improvement.

The retinal disease evaluation system of another aspect includes a retinal diseases evaluation program which enables a computer to function as storage means for storing vision-related quality of life (QOL) as evaluation information, and evaluation means for processing the evaluation information stored in the above storage means, and evaluating the presence or absence of retinal diseases, seriousness, or degree of improvement.

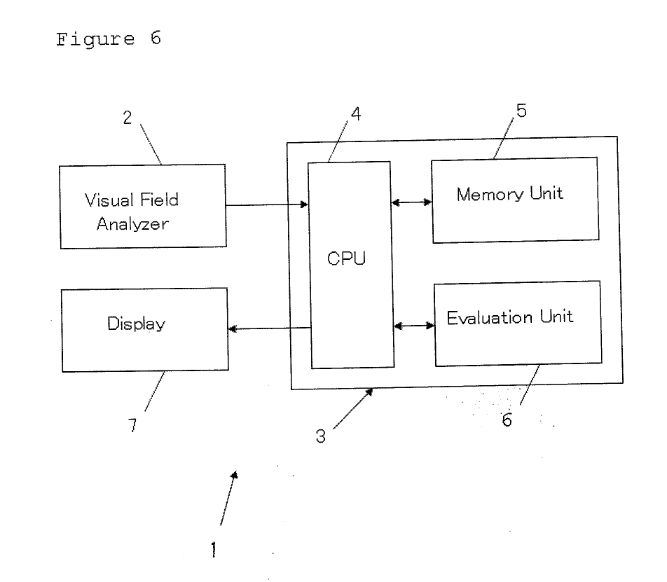

Referring to the accompanying drawings, discussions will be made to an embodiment of a system and method according to the present invention. FIG. 6 shows a diagram showing structural elements of the system for evaluating retinal diseases from retinal sensitivities. The evaluation system generally indicated by reference numeral 1 has a visual field analyzer 2. For example, the visual field analyzer 2 is a microperimeter which is commercially available from NIDEK Inc., 47651 Westinghouse Drive, Fremont, Calif. 94539-7474, under the trade-name “MP-1”, or a Humphrey visual field analyzer commercially available from Carl Zeiss Ophthalmic Systems, Inc., 5160 Hacienda Drive, Dublin, Calif. 94568, under the trade-name “Humphrey® Field Analyzer II-iseries”.

As is known well in the art, the visual field analyzer is designed to measure retinal sensitivities at the predetermined measurement points on fundus. For example, the visual field analyzer 2 measures the retina sensitivities at 24 points of central 10 degrees of the ocular fundus, at 12 points of central three degrees, or at four points of central two degrees in order to evaluate the presence or absence of retinal diseases, seriousness or severity level and degree of improvement or recovery/progress.

The system 1 also has a computer generally indicated by reference numeral 3 for processing the retina sensitivities measured by the analyzer 2 to evaluate the presence or absence of retinal diseases, seriousness and degree of improvement. The computer system can include one or more processors which can control the operation of the computer system. The processor(s) can include any type of microprocessor or central processing unit (CPU), including programmable general-purpose or special-purpose microprocessors. Conventional desktop computers, workstations, minicomputers, laptop computers, tablet computers, PDAs or other digital data processing apparatus of the type that are commercially available in the marketplace and that are suitable for operation in the illustrated system as described herein. The computer system can also include a memory, which can provide temporary or permanent storage for code/programs to be executed by the processor(s) or for data that is input to the computer system and/or acquired by the computer system. The memory can include read-only memory (ROM), flash memory, one or more varieties of random access memory (RAM), and/or a combination of memory technologies. The storage devices(s) can include any conventional medium for storing data in a non-volatile and/or non-transient manner. The storage device(s) can include one or more hard disk drives, flash drives, USB drives, optical drives, various media cards, and/or any combination thereof and can be directly connected to the computer system or remotely connected thereto, such as over a network. The elements illustrated in FIG. 6 can be some or all of the elements of a single physical machine. In addition, not all of the illustrated elements need to be located on or in the same physical machine. The computer system can be configured, either alone or in conjunction with other computer systems, to execute programs to perform any of the methods described herein or to perform certain steps of such methods. The programs can be stored on any of a variety of non-transitory computer-readable storage media, including hard disk drives, flash drives, USB drives, optical discs, media cards, memory systems, and/or combinations thereof. For this purpose, the computer 3 has a central processing unit (CPU) 4 which is electrically connected to an output of the visual field analyzer 2 to receive the retinal sensitivities measure by the analyzer 2. The CPU 4 is also connected to a memory means or memory unit 5 for memorizing the retinal sensitivities measured by and transmitted from the analyzer 2, and an evaluation means or unit 6 for evaluating the presence or absence of retinal diseases, seriousness (severity level) and degree of recovery and/or progress by the use of the retinal sensitivities. Preferably, the system 1 further has an output means or display 7 for visually showing the results made by the evaluation unit 6.

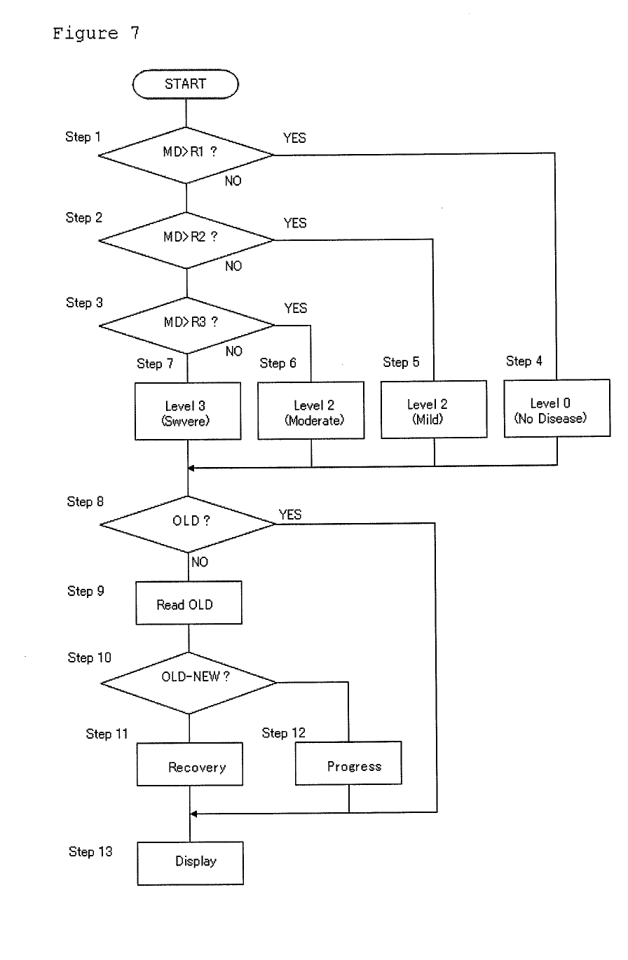

In operation of the system 1 so constructed, the retina sensitivities at 24 points of central 10 degrees of the ocular fundus, at 12 points of central three degrees, or at four points of central two degrees, measured by the analyzer 2 are transmitted to the computer 3 and memorized in the memory unit 5. The measurements are then transmitted to the evaluation unit 6 where they are processed in accordance with a program stored in a memory of the evaluation unit 6. Alternatively, the program may be stored in the memory unit 5. Specifically, as shown in FIG. 7, the evaluation unit 6 compares an average value MD (average) of the measured retinal sensitivities and one or more reference values R1, R2, and/or R3 (R1>R2>R3) stored in the memory of the evaluation unit 6 to determine the presence of retinal disease and/or a severity level (Level 0, 1, 2, or 3) of retinal disease of the patient (Steps 1 to 7). Then, the evaluation unit 6 determines whether the previously evaluated severity level of retinal disease of the patient is stored in the memory unit 5 or the memory of the evaluation unit 6 (Step 8). If the decision is affirmative (YES at Step 8), the evaluation unit 6 reads the previously evaluated severity level (OLD) (Step 9) and compares it with the newly determined evaluated severity level (NEW) obtained at previous steps 4, 5, 6, or 7 (Step 10). As a result of comparison, if the newly determined evaluated severity level (NEW) is lower than the previously evaluated severity level (OLD), a degree of recovery from retinal disease by, for example, using a difference between the newly and previously evaluated severity levels (Step 11). Contrary to this, if the newly determined evaluated severity level (NEW) is higher than the previously evaluated severity level (OLD), a degree of progress in retinal disease by, for example, using a difference between the newly and previously evaluated severity levels (Step 12). Although not shown, the newly evaluated severity level (NEW) is stored in the memory unit 5 or the memory of the evaluation unit 6. The presence/absence of retinal disease, the newly evaluated severity level (NEW), the previously evaluated severity level (OLD), the degree of recovery, and/or the degree of progress is transmitted to the display means or unit 7 and then indicated on the screen of the display unit 7 (Step 13).

The description of the invention is merely exemplary in nature and, thus, variations that do not depart from the gist of the invention are intended to be within the scope of the invention. Such variations are not to be regarded as a departure from the spirit and scope of the invention. For example, the memory unit 5 may store another information such as vision-related quality of life (QOL) of patients. The vision-related quality of life (QOL) may be determined with the 25-Item National Eye Institute Visual Functioning Questionnaire (NEI VFQ-25), or with the vision-related social function (SF) concerning subclass of NEI VFQ-25. Then, the determined vision-related quality of life (QOL) may be used independently or in combination with the measured retinal sensitivities to evaluate the presence/absence of retinal disease, the severity level, the degree of recovery, and the degree of progress.

What is claimed is:

1. A method for diagnosing and/or evaluating the presence or absence, severity or degree of the improvement of a retinal disease in a subject, which comprises determining and/or evaluating circulatory parameters, retinal function, retina morphology, retinal sensitivity and/or visual relating quality of life (QOL).

2. The method of claim 1, wherein diagnosis and/or evaluation is performed by at least one device selected from the group consisting of: Humphrey visual field analyzer (HFA) and Microperimeter-1 (MP-1).

3. The method of claim 2, which comprises:
determining retinal sensitivity in the central area of an ocular fundus of the subject by the Humphrey visual field analyzer (HFA) or a Microperimeter (MP-1), and
diagnosing and/or evaluating the presence or absence, severity, or degree of the improvement of the retinal disease based on the determined retinal sensitivity.

4. The method of claim 3, wherein retinal sensitivity is determined using one or more of:
central 10 degrees of an ocular fundus determined with micro perimeter MP-1;
central 3 degrees of an ocular fundus determined with micro perimeter MP-1;
central 2 degrees of an ocular fundus determined with micro perimeter MP-1;
central 10 degrees of an ocular fundus determined with Humphrey visual field analyzer;
central 3 degrees of an ocular fundus determined with Humphrey visual field analyzer; and
central 2 degrees of an ocular fundus determined with Humphrey visual field analyzer;

5. The method of claim 1, which comprises:
evaluating visual relating quality of life (QOL) of the subject and
diagnosing and/or evaluating the presence or absence, severity, or degree of the improvement of the retinal disease based on the visual relating quality of life (QOL).

6. The method of claim 5, wherein the vision related QOL is evaluated with “The 25-Item National Eye Institute Visual Function Questionnaire (NEI VFQ-25)”.

7. The method of claim 5, wherein the visual relating QOL is evaluated with the visual relating social function (SF)-concerning subclass of NEI VFQ-25.

8. The method of claim 1, wherein the diagnosis and/or evaluation is conducted by using a computer program for use with a computer, comprising:
a program instruction for causing a memory of the computer to store a information of detected and/or evaluated circulatory parameters, retinal function, retina morphology, retinal sensitivity and/or visual relating quality of life (QOL) of a subject as stored measurement information; and
a program instruction for causing an evaluation means of the computer to process the stored measurement information and evaluate presence or absence, severity or degree of improvement of a retinal disease in the subject.

9. A method for evaluating the effectiveness of a test compound for the treatment of retinal disease, which comprises:
(i) detecting and/or evaluating circulatory parameters, retinal function, retina morphology, retinal sensitivity and/or visual relating quality of life (QOL) of the subject to obtain a baseline value,
(ii) administering to the subject a composition comprising a test compound,
(iii) detecting and/or evaluating circulatory parameters, retinal function, retina morphology and/or visual relating quality of life (QOL) of the subject after the administration of the composition comprising a test compound to obtain a test value, and
(iv) comparing the baseline value to the test value, wherein the difference between the baseline value and test value indicates the effectiveness of the test compound.

10. A method for treating a retinal disease in a subject, comprising:
measuring the central area of an ocular fundus in a subject to determine one or more of circulatory parameters, retinal function, retina morphology and retinal sensitivity,
diagnosing and/or evaluating the presence, severity, or degree of the improvement of the retinal disease based on the determined circulatory parameters, retinal function, retina morphology and/or retinal sensitivity in the subject, and
adjusting the dosing or treatment protocol based on the presence, severity, or degree of the improvement of the retinal disease.

11. The method of claim 10, wherein retinal sensitivity in the central area of an ocular fundus of the subject is determined using at least central 2 degrees, and the presence, severity, or degree of the improvement of the retinal disease is diagnosed and/or evaluated based on the determined retinal sensitivity in the subject.

12. The method of claim 11, wherein the presence, severity, or degree of the improvement of the retinal disease is diagnosed and/or evaluated based on one or more of the circulatory parameters, retinal function and retina morphology, in addition to the retinal sensitivity in the subject.

13. The method of claim 10, which further comprising:
evaluating visual relating quality of life (QOL) of the subject,
wherein the presence, severity, or degree of the improvement of the retinal disease is determined and/or evaluated based on the QOL in addition to one or more of the circulatory parameters, retinal function, retina morphology and retinal sensitivity in the subject.
